(19) United States
(12) Patent Application Publication
(10) Pub. No.: US 2022/0008445 A1
(43) Pub. Date: Jan. 13, 2022

(54) METHODS, COMPOSITIONS, AND KITS FOR TREATING OCULAR DISEASES

(71) Applicant: ELOXX PHARMACEUTICALS, Waltham, MA (US)

(72) Inventors: John VAN DUZER, Waltham, MA (US); Greg WILLIAMS, Waltham, MA (US)

(21) Appl. No.: 17/296,203

(22) PCT Filed: Nov. 27, 2019
(86) PCT No.: PCT/US2019/063781

§ 371 Date: May 21, 2021

Related U.S. Application Data
(60) Provisional application No. 62/773,131, filed on Nov. 29, 2018.
(60) Provisional application No. 62/783,852, filed on Dec. 21, 2018.
(60) Provisional application No. 62/838,905, filed on Apr. 25, 2019.
(60) Provisional application No. 62/878,260, filed on Jul. 24, 2019.

(51) Int. Cl. A61K 31/7036; A61K 9/00

(52) U.S. Cl. CPC A61K 31/7036; A61K 9/0019; A61K 9/0048

(57) ABSTRACT

Provided herein are methods of treating an ocular disease associated with one or more nonsense mutation in a subject using one or more amino-glycosides and/or derivatives thereof. The pharmaceutical compositions and kits containing one or more aminoglycosides and/or derivatives thereof for treating the ocular disease are disclosed.

### PRIORITY CLAIM

This application claims priority to U.S. Provisional Appl. No. 62/773,131, filed on Nov. 29, 2018; U.S. Provisional Appl. No. 62/783,852, filed on Dec. 21, 2018; U.S. Provisional Appl. No. 62/838,905, filed on Apr. 25, 2019 and U.S. Provisional Appl. No. 62/878,260, filed on Jul. 24, 2019, all of which are incorporated herein by reference in their entireties.

### BACKGROUND

A number of ocular diseases are associated with nonsense mutations, which cause premature termination of gene expression. For example, retinitis pigmentosa (RP) causes vision loss and affects about one out of every 3,500 people in the U.S. and Europe. 15% of all occurrences of RP are caused by nonsense mutations.

RP is responsible for the vision loss component of Usher syndrome, the most common form of deaf-blindness worldwide. Usher syndrome affects 3.2-6.2 per 100,000 people, with about 16,300 cases in the U.S. There are three clinical types of Usher Syndrome (USH1, USH2, USH3), and twelve genetic loci have been described: USH1B-H; USH2A, C, D; and USH3A. The ratio of USH2 to USH1 is about 3:2. Nonsense mutations account for approximately 20% of all Usher Syndrome cases.

Aniridia is a fundamental disturbance in the development of the eye, with a prevalence of approximately 1.8 per 100,000 people, affecting about 5,900 people in the U.S. Nonsense mutations account for about 40% of all cases of aniridia.

Choroideremia is a slowly progressing vision loss and affects about 1-2 per 100,000 people and about 6,500 people in the U.S. Nonsense mutations account for about 40% of the genetic changes.

Stickler Syndrome has a prevalence of about 1-3 per 100,000 people, affecting about 66,000 people in the U.S. Nonsense mutations account for about 20% of all cases of Stickler Syndrome.

Leber congenital amaurosis (LCA) has a prevalence of about 1-9 per 100,000 people, and accounts for approximately 5% of all retinal dystrophies.

There are currently no drugs approved or in late-stage development that target nonsense mutations associated with inherited retinal diseases. Thus, there is a significant unmet need in the art for improved methods of treating Usher Syndrome, RP, aniridia, choroideremia, Stickler Syndrome, LCA, and other ocular diseases associated with nonsense mutations.

### SUMMARY

Provided herein in certain embodiments are methods of treating an ocular disease associated with one or more nonsense mutations in a subject comprising administering to said subject one or more aminoglycosides and/or derivatives thereof. In certain of these embodiments, the one or more aminoglycosides and/or derivatives thereof are administered as part of a pharmaceutical formulation comprising the one or more aminoglycosides and/or derivatives thereof and, optionally, one or more pharmaceutically acceptable carriers.

Provided herein in certain embodiments are methods of increasing gene expression or gene read-through in a retinal cell comprising administering to said subject one or more aminoglycosides and/or derivatives thereof. In certain of these embodiments, the gene whose expression or read-through is being increased comprises one or more nonsense mutations. In certain embodiments, the one or more aminoglycosides and/or derivatives thereof are administered as part of a pharmaceutical formulation comprising the one or more aminoglycosides and/or derivatives thereof and, optionally, one or more pharmaceutically acceptable carriers.

Provided herein in certain embodiments, administering one or more aminoglycosides and/or derivatives thereof induces read-through activity without exceeding the safety exposure threshold of the one or more aminoglycosides and/or derivatives.

Provided herein in certain embodiments are one or more aminoglycosides and/or derivatives thereof, or pharmaceutical formulations comprising one or more aminoglycosides and/or derivatives thereof, to treat an ocular disease associated with one or more nonsense mutations.

Provided herein in certain embodiments are one or more aminoglycosides and/or derivatives thereof, or pharmaceutical formulations comprising one or more aminoglycosides and/or derivatives thereof, to increase gene expression or gene read-through in an ocular cell.

Provided herein in certain embodiments is the use of one or more aminoglycosides and/or derivatives thereof, or of a pharmaceutical formulation comprising one or more aminoglycosides and/or derivatives thereof, to treat an ocular disease associated with one or more nonsense mutations.

Provided herein in certain embodiments is the use of one or more aminoglycosides and/or derivatives thereof, or of a pharmaceutical formulation comprising one or more aminoglycosides or derivatives thereof, to increase gene expression or gene read-through in a retinal cell.

Provided herein in certain embodiments is the use of one or more aminoglycosides and/or derivatives thereof to formulate a medicament for treating an ocular disease associated with one or more nonsense mutations.

Provided herein in certain embodiments is the use of one or more aminoglycosides and/or derivatives thereof to formulate a medicament for increasing gene expression or gene read-through in a retinal cell.

Provided herein in certain embodiments are kits comprising one or more aminoglycosides and/or derivatives thereof or pharmaceutical formulations comprising one or more aminoglycosides and/or derivatives thereof for use in treating an ocular disease associated with one or more nonsense mutations. In certain of these embodiments, the kits further comprise instructions for use.

Provided herein in certain embodiments are kits comprising one or more aminoglycosides and/or derivatives thereof or pharmaceutical formulations comprising one or more aminoglycosides and/or derivatives thereof for use in increasing gene expression or gene read-through in a retinal cell. In certain of these embodiments, the kits further comprise instructions for use.

In certain of embodiments of the methods and uses provided herein, the one or more aminoglycosides and/or derivatives thereof, or pharmaceutical formulations thereof, are administered in vitro or in vivo. In certain embodiments, the one or more aminoglycosides and/or derivatives thereof, or pharmaceutical formulations thereof, are administered to a subject via topical or intravitreal administration.

In certain embodiments of the methods, uses, formulations, and kits provided herein, the ocular disease associated with one or more nonsense mutations is selected from the group consisting of Usher Syndrome, RP, Stickler Syndrome, aniridia, LCA, or choroideremia. In certain embodiments wherein the ocular disease is Usher Syndrome, the nonsense mutation may be in one or more of USH1B-G, USH2A or C-D, or USH3A.

In certain embodiments, the methods, uses, formulations, and kits provided herein, the one or more nonsense mutations are selected from the group consisting of R3X (PCDH11), R155X (USH1C), R245X (PCDH15), and R626X (USH2A).

In certain embodiments of the methods, uses, pharmaceutical formulations, and kits provided herein, the one or more aminoglycosides and/or derivatives thereof are selected from the Category I-IV Compounds described below. In certain of these embodiments, the one or more aminoglycosides and/or derivatives thereof are selected from the group consisting of NB30, NB54, NB84, NB118, NB122, NB124, NB124-MeS, NB127, NB128, and NB157.

In certain embodiments of the methods provided herein, the methods further comprise administering one or more additional therapeutic agents in combination with the one or more aminoglycosides or derivatives thereof, or pharmaceutical formulations thereof. These additional therapeutic agents may be administered in the same or in different formulations. In certain embodiments of the methods provided herein, 0.3 mg/kg to about 2.5 mg/kg of the one or more aminoglycosides is administered to the subject.

### BRIEF DESCRIPTION OF THE DRAWINGS

FIG. 1 shows Usher Syndrome variants.

FIG. 2 shows the plots for a dark-adapted oscillatory potential and photopic b-wave amplitudes measured pre and post-9 days after administration of NB118 to rabbits.

FIG. 3 shows the H&E stained sections of rabbits treated with gentamicin and NB118.

FIG. 4 shows a plot of the pharmacokinetic profile of NB122.

FIGS. 5A-B shows cell-free read-through of USH1 nonsense mutations in the presence of NB84 and NB127.

FIGS. 6A-B shows in vitro read-through of USH1 nonsense mutations in the presence of NB84 and NB127.

FIG. 7 shows the read-through activity of NB122, NB84, NB127, NB118, NB128, NB124-MeS, and NB157 against the R626XUSH2A mutation.

FIG. 8 shows the read-through activity of NB122, NB84, NB127, NB118, NB128, NB124-MeS, and NB157 against the R3XUSH1F mutation.

FIG. 9 shows the comparative read-through activity of NB122, NB84, NB127, NB118, NB128, NB124-MeS, and NB157 against the R626XUSH2A and R3X USH1F mutations.

FIG. 10 shows the read-through activity of NB122, NB125, and NB84 against the R626XUSH2A mutation.

FIG. 11 shows the read-through activity of NB122, NB125, and NB84 against the R3XUSH1F mutation.

FIG. 12 shows a scheme for evaluating read-through activity of aminoglycosides on a R213X nonsense mutation of human TP53.

FIG. 13 shows representative western blots for the detection of full length p53 after administration of NB84 and NB128.

FIG. 14 shows a plot quantifying p53 after administration of NB84 and NB128 as compared to LaminB1.

FIG. 15 shows an image of a high-throughput immunofluorescence assay measuring p53 localized at the nucleus and the dose-dependence of NB84.

FIG. 16 shows full dose response curves for NB122, NB124, NB84, NB118, and NB128 as compared to gentamicin and vehicle control.

FIG. 17 shows Usher protein production in the presence of NB84, NB122, NB124, and NB127.

FIG. 18 shows a schematic of an intravitreal injection of SJL/mice.

FIG. 19 shows the results from a pilot study on the in vivo read-through of gentamicin.

FIG. 20 shows the dose dependent increase in melanin production in the eye upon administration of NB122, NB124, NB84, NB118, and NB128.

FIG. 21 shows the melanin production from the intravitreal dosing of NB122, NB84, NB127, NB118, NB128, NB124-MeS, and NB157 in mice models.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### Definitions

The terms “treat,” “treating,” and “treatment” as used herein with regard to an ocular disease associated with one or more nonsense mutations may refer to eliminating this disease; preventing, delaying, or reducing the likelihood of development or progression of this disease or of one or more symptoms associated with this disease; reducing or eliminating one or more symptoms associated with this disease; reducing the severity or occurrence of one or more symptoms associated with this disease; or some combination thereof.

A “subject” as used herein refers to a mammalian subject, preferably a human, who has been diagnosed with, is suspected of having, is at risk of developing, or is exhibiting or has exhibited one or more symptoms associated with an ocular disease associated with one or more nonsense mutations. In certain embodiments, the subject suffers from or is at risk of suffering from Usher Syndrome, RP, Stickler Syndrome, aniridia, LCA, or choroideremia.

A “therapeutically effective amount” of an aminoglycoside or a derivative thereof as used herein is an amount of the aminoglycoside or a derivative thereof that produces a desired therapeutic effect in a subject. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. The precise therapeutically effective amount for a particular aminoglycoside or a derivative thereof will vary based on a variety of factors, including but not limited to the characteristics of the aminoglycoside or a derivative thereof (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers present in the aminoglycoside composition, and the route of administration. One skilled in the clinical and pharmacological arts will be able to determine a therapeutically effective amount through routine experimentation, namely by monitoring a subject's response to administration of the aminoglycoside or a derivative thereof and adjusting the dosage accordingly. For additional guidance, see, e.g., Remington: The Science and Practice of Pharmacy, 22^{nd} Edition, Pharmaceutical Press, London, 2012, and Goodman & Gilman's The Pharmacological Basis of Therapeutics, 12^{th} Edition, McGraw-Hill, New York, N.Y., 2011, the entire disclosures of which are incorporated by reference herein.

The term “monosaccharide” as used herein and as well known in the art, refers to a simple form of a sugar that consists of a single saccharide molecule which cannot be further decomposed by hydrolysis. Most common examples of monosaccharides include glucose (dextrose), fructose, galactose, and ribose. Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms. Monosaccharides are the building blocks of oligosaccharides like sucrose (common sugar) and other polysaccharides (such as cellulose and starch).

The term “oligosaccharide” as used herein refers to a compound that comprises two or more monosaccharide units, as these are defined herein, linked to one another via a glycosyl bond (—O—). Preferably, the oligosaccharide comprises 2-6 monosaccharides, more preferably the oligosaccharide comprises 2-4 monosaccharides and most preferably the oligosaccharide is a disaccharide moiety, having two monosaccharide units.

The phrase “pharmaceutically acceptable carrier” as used herein refers to a pharmaceutically acceptable material, composition, or vehicle that is involved in carrying or transporting a compound or molecule of interest from one tissue, organ, or portion of the body to another tissue, organ, or portion of the body. A pharmaceutically acceptable carrier may comprise a variety of components, including but not limited to a liquid or solid filler, diluent, excipient, solvent, buffer, encapsulating material, surfactant, stabilizing agent, binder, or pigment, or some combination thereof. Each component of the carrier must be “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits.

The terms “compound” or “compounds” refer to conventional chemical compounds (e.g., small organic). As used herein, the phrase “small molecule” is used interchangeably with the term “compound.”

The term “about” as used herein means within 10% of a stated value or range of values.

The phrase “therapeutically effective amount” as used herein is an amount of the agent that produces a desired therapeutic effect in a subject.

The term “alkyl” as used herein refers to an aliphatic hydrocarbon including straight chain and branched chain groups. The alkyl may have 1 to 20 carbon atoms, or 1-10 carbon atoms, and may be branched or unbranched. According to some embodiments of the present invention, the alkyl is a low (or lower) alkyl, having 1-4 carbon atoms (namely, methyl, ethyl, propyl and butyl).

The terms substituted “alkyl,” “cycloalkyl,” “aryl,” “alkylaryl,” “heteroaryl,” “heteroalicyclic,” “acyl,” include but are not limited to hydroxy, alkoxy, thiohydroxy, thioalkoxy, aryloxy, thioaryloxy, alkaryl, alkenyl, alkynyl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide.

The term “solvate” as used herein refers to a complex of variable stoichiometry (e.g., di-, tri-, terra-, penta-, hexa-, and so on), which is formed by a solute (the compound of the present invention) and a solvent, whereby the solvent does not interfere with the biological activity of the solute. Suitable solvents include, for example, ethanol, acetic acid and the like.

The terms “hydroxyl” or “hydroxy” as used herein refer to an —OH group.

The term “amine” as used herein refers to a —NR′R″ group where each of R′ and R″ is independently hydrogen, alkyl, alkenyl, alkynyl, cycloalkyl, heteroalicyclic, aryl, heteroaryl, alkaryl, alkheteroaryl, or acyl, as these terms are defined herein. Alternatively, one or both of R′ and R″ can be, for example, hydroxy, alkoxy, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

A numerical range; e.g., “1-10”, as used herein, implies that the group, for example, an alkyl group, may contain 1 carbon atom, 2 carbon atoms, 3 carbon atoms, etc., up to and including 40 carbon atoms or more.

The phrases “substituted alkyl” or “substituent alkyl” as used herein refer to one or more of an alkyl (e.g., forming a branched alkyl), an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow. An alkyl substituted by aryl is also referred to herein as “alkaryl”, an example of which is benzyl.

The term “alkenyl” as used herein refers to an unsaturated alkyl for example, having at least two carbon atoms and at least one carbon-carbon double bond, e.g., allyl, vinyl, 3-butenyl, 2-butenyl, 2-hexenyl and i-propenyl. The alkenyl may be substituted or unsubstituted by one or more substituents.

The term “alkynyl” as used herein refers to an unsaturated alkyl having for example, at least two carbon atoms and at least one carbon-carbon triple bond. The alkynyl may be substituted or unsubstituted by one or more substituents.

The term “cycloalkyl” as used herein refers to an all-carbon monocyclic or fused ring (i.e., rings which share an adjacent pair of carbon atoms), branched or unbranched group containing 3 or more carbon atoms where one or more of the rings does not have a completely conjugated pi-electron system, and may further be substituted or unsubstituted. Exemplary cycloalkyl groups include, for example, cyclopropyl, cyclobutyl, cyclopentyl, cyclohexyl, or cyclododecyl. The cycloalkyl can be substituted or unsubstituted. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl.

The term “aryl” as used herein refers to an all-carbon monocyclic or fused-ring polycyclic (i.e., rings which share adjacent pairs of carbon atoms) groups having a completely conjugated pi-electron system. The aryl group may be unsubstituted or substituted by one or more substituents. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl.

The term “heteroaryl” as used herein refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system.

The term “heteroalicyclic” as used refers to a monocyclic or fused ring group having in the ring(s) one or more atoms such as nitrogen, oxygen and sulfur. The rings may also have one or more double bonds. However, the rings do not have a completely conjugated pi-electron system.

The term “oxo” as used herein, describes a (=0) group, wherein an oxygen atom is linked by a double bond to the atom (e.g., carbon atom) at the indicated position.

The term “hydroxyalkyl” as used herein refers to an alkyl group, as defined herein, substituted with one or more hydroxy group(s), e.g., hydroxymethyl, 2-hydroxyethyl and 4-hydroxypentyl.

### Treatment of Ocular Disease

Nonsense mutations cause premature translational termination, which leads to truncated protein products that are typically unstable. In the context of the ocular diseases being treated herein, these nonsense mutations result in various deleterious effects, including progressive photoreceptor loss. The aminoglycosides and derivatives thereof disclosed herein can be used as small molecule drugs to facilitate read-through of premature stop codons, extending mRNA half-life and restoring full-length functional proteins.

As disclosed herein, aminoglycosides including NB30/Compound 3, NB54/Compound 37, NB84/ELX-03, NB118/ELX-05, NB122/ELX-01, NB124/ELX-02, NB124-MeS, NB127/ELX-04, NB128, and NB157/ELX-06 have been found to demonstrate favorable safety profiles in the retina, read-through of relevant eye-disorder mutations, and/or production of the missing protein due to nonsense mutations. Based on these findings, methods, uses, formulations, and kits are provided for treating ocular diseases associated with one or more nonsense mutations using one or more aminoglycosides and/or derivatives thereof.

Ocular diseases associated with nonsense mutations that may be treated using the methods, uses, formulations, and kits provided herein include, but are not limited to, Usher Syndrome, retinitis pigmentosa, Stickler Syndrome, Stargardt's disease, aniridia, LCA, achromatopsia, and choroideremia. The ocular disease may be another inherited retinal disease.

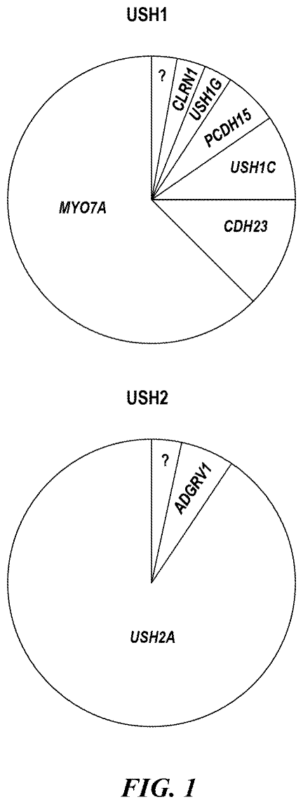

Usher syndrome has multiple variants, some of which are illustrated in FIG. 1. Nonsense mutations are relatively common across all subtypes, with an overall prevalence of about 20%. Accordingly, in certain embodiments of the methods, uses, formulations, and kits disclosed herein, the ocular disease associated with a nonsense mutation is Usher Syndrome. In these embodiments, the nonsense mutations may be located in one or more of Usher 1 (USH1), Usher 2 (USH2) or Usher 3 (USH3) subtypes. In certain embodiments, the nonsense mutation is selected from the group consisting of R3X (PCDH11), R155X (USH1C), R245X (PCDH15), and R626X (USH2A). In other embodiments, the nonsense mutation is a USH2A mutation R626X

As disclosed herein, the aminoglycosides NB30, NB54, NB84, NB118, NB122, NB124, NB124-MeS, NB127, NB128, and NB157 were shown to promote read-through of multiple common nonsense mutations associated with Usher Syndrome. For example, NB84 and NB127 induced significant read-through as compared to G418 and gentamicin in both cell-free and in vitro assays of the nonsense mutations R3X and R245X in the PCDH11 gene. NB84, NB127, NB118, NB128, NB124-MeS, and NB157 also induced significant read-through in in vitro cell-based assays based on nonsense mutations located in both USH1 and USH2 subtypes, R3X and R626X, respectively.

As disclosed herein, the aminoglycosides were shown to promote read-through of multiple nonsense mutations associated with Usher syndrome by increased protein expression. For example, NB84, NB122, NB124, and NB127 were shown to increase protein expression adversely affected by the nonsense mutation R155X located on the USH1C subtype.

As disclosed herein, the aminoglycosides were shown to induce read-through at permissive concentrations by intravitreal injection. NB122, NB124, NB124-MeS, NB84, NB118, NB127, NB128, and NB147 showed significant read-through as compared to gentamicin upon injection to the posterior segment (i.e., back of eye) in in vivo studies. With the USH1F mutation R3X, NB122 demonstrated a 2.5-fold increase in read-through versus native read-through, which represents a 2.7% R3X read-through rate. Intravitreal injection NB122, NB124, NB124-MeS, NB84, NB118, NB127, NB128, and NB147 of preclinical models were also found to be well-tolerated in comparison to gentamicin in both gross ophthalmological examination and electroretinogram. In addition, intravitreal injection was sufficient to induce read-through at permissive concentrations of NB122, NB124, NB124-MeS, NB84, NB118, NB127, NB128, and NB147 within the retina.

Accordingly, provided herein in certain embodiments are methods of treating ocular conditions associated with nonsense mutations, including Usher Syndrome, by administering a therapeutically effective amount of one or more aminoglycosides and/or derivatives thereof, or a pharmaceutical formulation comprising one or more aminoglycosides and/or derivatives thereof.

Provided herein in certain embodiments are methods of treating an ocular disease associated with one or more nonsense mutations in a subject, comprising administering to said subject a therapeutically effective amount of one or more aminoglycosides and/or derivatives thereof selected from the group consisting of NB30, NB54, NB84, NB118, NB122, NB124, NB124-MeS, NB127, NB128, and NB157. In some embodiments, the one or more nonsense mutations are selected from the group consisting of R3X, R155X, R245X, and R626X In some embodiments the one or more aminoglycosides and/or derivatives thereof are administered intravitreally.

Provided herein in certain embodiments are methods of treating ocular disease associated with one or more nonsense mutations in a subject, comprising administering to said subject a therapeutically effective amount of one or more aminoglycosides, wherein said one or more nonsense mutations are selected from the group consisting of R3X, R155X, R245X, and R626X In some embodiments, the one or more aminoglycosides and/or derivatives thereof are selected from the group consisting of NB30, NB54, NB84, NB118, NB122, NB124, NB124-MeS, NB127, NB128, and NB157. In some embodiments the one or more aminoglycosides and/or derivatives thereof are administered intravitreally.

Provided herein in certain embodiments are methods of increasing gene expression or gene read-through in a retinal cell comprising contacting the retinal cell with one or more aminoglycosides and/or derivatives thereof, or a pharmaceutical formulation comprising one or more aminoglycosides and/or derivatives thereof, wherein the gene expression or the gene read-through is adversely affected by one or more nonsense mutations. In certain embodiments, increasing gene expression or gene read-through increases a production of proteins adversely affected by Usher Syndrome, retinitis pigmentosa, Stickler Syndrome, Stargardt's disease, aniridia, LCA, achromatopsia, and choroideremia.

In certain embodiments of the methods provided herein, administration of the one or more aminoglycosides and/or derivatives thereof increases gene expression or gene read-through by at least about 0.5%, at least about 1%, at least about 1.5%, at least about 2%, at least about 2.5%, at least about 3%, at least about 3.5%, at least about 4%, at least about 5%, at least about 5.5%, at least about 6%, at least about 6.5%, at least about 7%, at least about 7.5%, at least about 8%, at least about 8.5%, at least about 9%, at least about 9.5%, at least about 10%, or more. For example, in some embodiments the gene expression or gene read through increases about 1% to about 5%, about 1% to about 10%, about 1% to about 3%, about 3% to about 6%, about 6% to about 10%, about 2.5% to about 3.5%, about 2% to about 10%, about 2.5% to about 6%, or about 3% to about 8%.

In certain embodiments of the methods provided herein, administration of the one or more aminoglycosides and/or derivatives thereof increases the production of proteins adversely affected by one or more nonsense mutations. In some embodiments, the protein production increases by at least about 0.5%, at least about 1%, at least about 1.5%, at least about 2%, at least about 2.5%, at least about 3%, at least about 3.5%, at least about 4%, at least about 5%, at least about 5.5%, at least about 6%, at least about 6.5%, at least about 7%, at least about 7.5%, at least about 8%, at least about 8.5%, at least about 9%, at least about 9.5%, at least about 10%, or more. For example, in some embodiments the gene expression or gene read through increases about 1% to about 5%, about 1% to about 10%, about 1% to about 3%, about 3% to about 6%, about 6% to about 10%, about 2.5% to about 3.5%, about 2% to about 10%, about 2.5% to about 6%, or about 3% to about 8%.

In certain embodiments of the methods provided herein, the one or more aminoglycosides and/or derivatives thereof are intravitreally administered to treat the ocular conditions associated with nonsense mutations. In some embodiments, the one or more aminoglycosides and/or derivatives thereof or pharmaceutical formulation comprising one or more aminoglycosides and/or derivatives thereof are formulated for injection into a posterior segment the subject's eye (e.g., back of eye). In some embodiments, the intravitreal injection comprises administering a therapeutically effective amount of one or more aminoglycosides and/or derivatives thereof to treat the ocular condition associated with nonsense mutations.

In certain embodiments the methods of treating ocular conditions associated with nonsense mutations comprises administering the one or more aminoglycosides and/or derivatives thereof in an amount that does not exceed the safety exposure threshold of the one or more aminoglycosides and/or derivatives thereof. In some embodiments, the safety exposure threshold of the one or more aminoglycosides and/or derivatives thereof is greater than or equal to the read-through threshold for the one or more aminoglycosides and/or derivatives thereof. In some embodiments, administration of the one or more aminoglycosides and/or derivatives thereof induces read-through without exceeding the exposure safety threshold.

In certain embodiments the methods of treating ocular conditions associated with nonsense mutations comprises intravitreally administering the one or more aminoglycosides and/or derivatives thereof to a subject in need thereof, wherein the subject exhibits no serious irreversible eye damaged and/or complications. In some embodiments, the subject exhibits no adverse structural changes to the eye upon intravitreally injection of the one or more aminoglycosides and/or derivatives thereof.

In certain embodiments of the methods provided herein, a single aminoglycoside or derivative thereof, or a pharmaceutical formulation comprising a single aminoglycoside and/or derivative thereof, is administered. In other embodiments, two or more aminoglycosides and/or derivatives thereof, or a pharmaceutical formulation comprising two or more aminoglycosides and/or derivatives thereof, are administered. In certain embodiments, one or more additional therapeutic agents are administered in addition to the one or more aminoglycosides and/or derivatives thereof. The one or more additional therapeutic agents may be administered as part of the same formulation as the one or more aminoglycosides and/or derivatives thereof, or they may be administered separately.

In certain embodiments of the methods provided herein, the one or more aminoglycosides and/or derivatives thereof may be administered for a specific time course determined in advance. For example, the one or more aminoglycosides and/or derivatives thereof may be administered for a time course of 2 weeks, 4 weeks, 6 weeks, 8 weeks, 12 weeks, 16 weeks, 20 weeks, 24 weeks, 36 weeks, 48 weeks, 1 year, 18 months, 2 years, or more than 2 years. In other embodiments, the one or more aminoglycosides and/or derivatives thereof may be administered indefinitely, or until a specific therapeutic benchmark is reached. For example, the one or more aminoglycosides and/or derivatives thereof may be administered until one or more symptoms of the ocular disease have been eliminated or reduced to a desired level.

Provided herein in certain embodiments is the use of one or more aminoglycosides and/or derivatives thereof for formulating a medicament for treating an ocular disease associated with one or more nonsense mutations.

Provided herein in certain embodiments are one or more aminoglycosides and/or derivatives thereof, or pharmaceutical formulations comprising one or more aminoglycosides and/or derivatives thereof, for use in treating an ocular disease associated with one or more nonsense mutations.

### Aminoglycosides and Derivatives Thereof

In certain embodiments of the methods, uses, compositions, and kits provided herein, the one or more aminoglycosides and/or derivatives thereof are selected from the group consisting of Category I compounds, Category II compounds, Category III compounds, Category IV compounds, and Category VI compounds as described below. In certain embodiments, the aminoglycosides or derivatives thereof are selected from the group consisting of NB30, NB54, NB84, NB118, NB122, NB124, NB124-MeS, NB127, NB128, and NB157. In other embodiments, the aminoglycosides and/or derivatives thereof are gentamicin X2. In certain embodiments, the aminoglycosides and derivatives thereof disclosed herein have reduced affinity to prokaryotic ribosomes and preferentially bind to eukaryotic ribosomes.

### Category I Compounds

Category I compounds include all aminoglycosides disclosed or claimed in any application or patent claiming priority to U.S. Provisional Appl. No. 60/788,070, filed Apr. 3, 2006, and/or PCT Appl. No. PCT/IL2007/000463, filed Apr. 10, 2007 (PCT Publ. No. WO07/113841). These include, but are not limited to, U.S. Pat. Nos. 9,073,958 and 9,821,001; CA Patent No. 2,646,407; and EP Patent Nos. 2007783 and 2390255.

Examples of Category I compounds include, without limitation, compounds of Formula I:

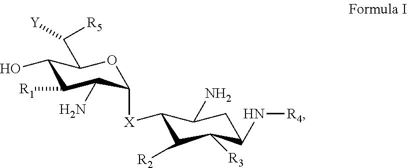

or stereoisomers or pharmaceutically acceptable salts thereof, wherein:

each of R1, R2, and R3 is independently a monosaccharide moiety, halide, hydroxyl, amine, or oligosaccharide moiety;

X is oxygen or sulfur;

R₄ is hydrogen or (S)-4-amino-2-hydroxybutyryl (AHB);

R₅ is hydroxyl;

Y is hydrogen or alkyl; and

the dashed line indicates an R configuration or an S configuration,

with the proviso that the compound is not selected from the group consisting of

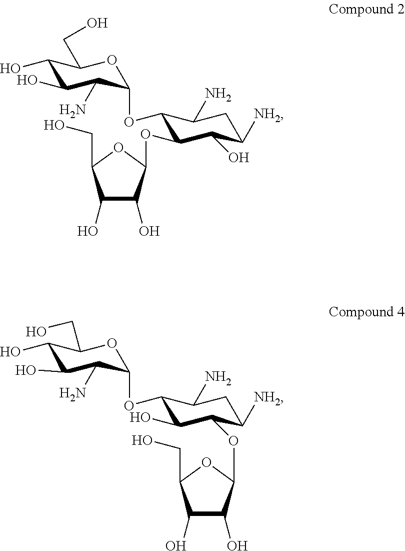

amikacin, apramycin, arbekacin, butirosin, dibekacin, fortimycin, G-418, gentamicin, hygromycin, habekacin, dibekacin, netlmicin, istamycin, isepamycin, kanamycin, lividomycin, neamine, neomycin, paromomycin, ribostamycin, sisomycin, spectinomycin, streptomycin, and tobramycin.

In certain embodiments, a Category I compound having Formula I is selected from the group consisting of:

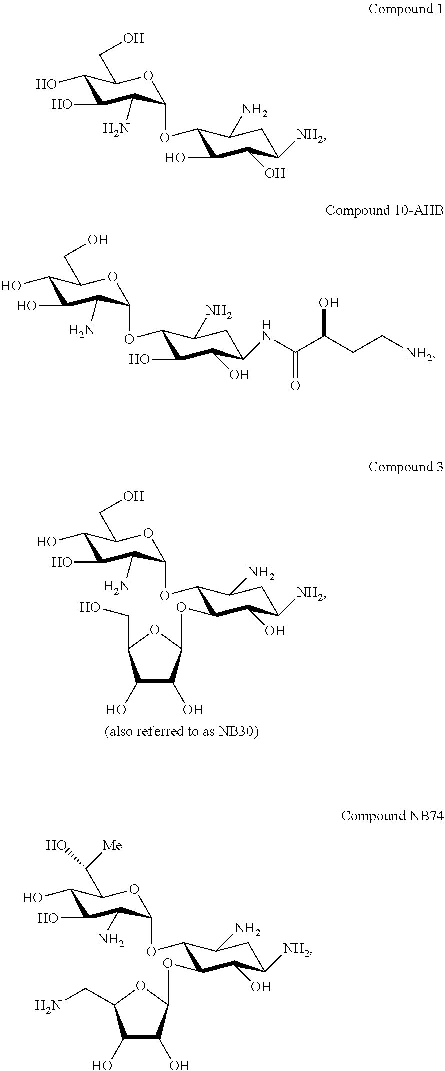

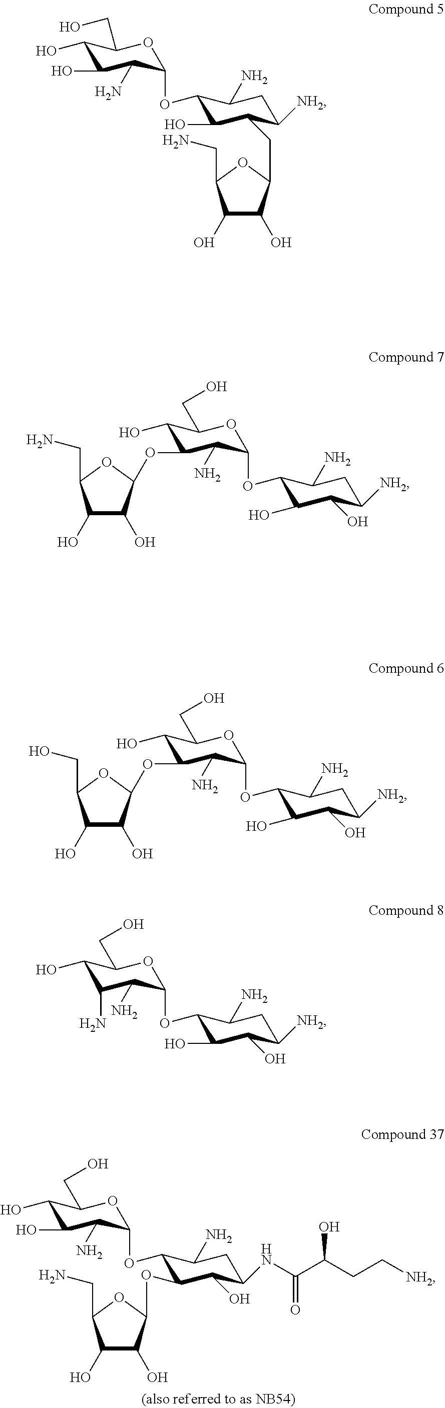

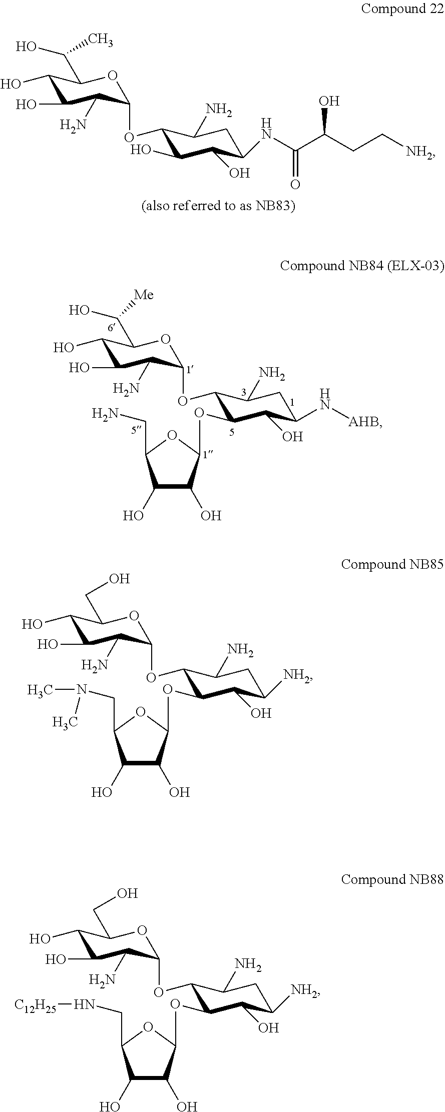

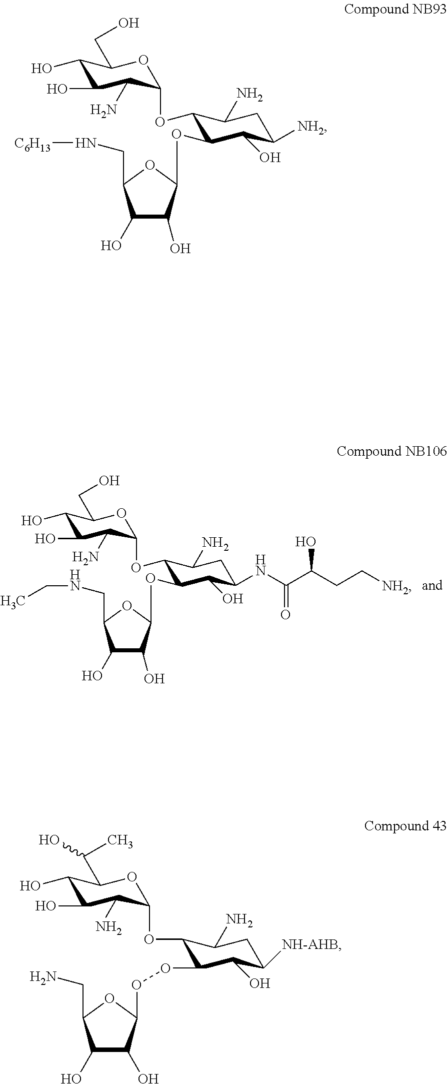

or a stereoisomer or pharmaceutically acceptable salt thereof.

In certain embodiments wherein one or more of R₁, R₂, and R₃ is a monosaccharide moiety, the monosaccharide moiety has the structure set forth in Formula II:

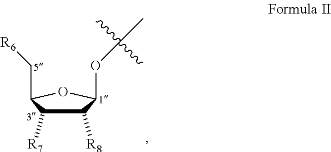

wherein:

the dashed line indicates an R configuration or an S configuration; and

each of R₆, R₇, and R₈ is independently selected from the group consisting of hydroxyl and amine.

In some embodiments, the amine is a substituted or unsubstituted amine. In some embodiments, the amine is an alkyl substituted amine (e.g., N(CH₃)₂, NH(C₁₂H₂₅), NH(C₆H₁₃), or NH(CH₂CH₃)).

Additional examples of Category I compounds include, without limitation, compounds of Formula Ia*:

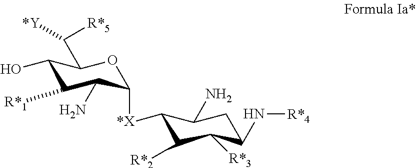

or stereoisomers or pharmaceutically acceptable salts thereof, wherein:

the dashed line indicates an R configuration or an S configuration;

each of R*₁, R*₂, and R*₃ is independently a halide, hydroxyl, amine, or is linked to the compound having Formula I, wherein at least one of R*₁, R*₂ and R*₃ is linked to the compound having Formula I;

X* is oxygen or sulfur;

R*₄ is hydrogen or an AHB moiety;

R*₅ is hydroxyl or amine; and

Y* is hydrogen, alkyl or aryl.

In certain embodiments, a Category I compound is a dimer comprising two units of Formula Ia* attached via their corresponding R₁, R*₁, R₂, R*₂, R₃, or R*₃ positions in any combination thereof, for example, an R₁-R*₂ or R₂-R*₁ linked dimer, an R₁-R*₃ or R₃-R*₁ linked dimer, an R₃-R*₂ or R₂—R*₃ linked dimer, an R₁-R*₁ linked dimer, an R₂-R*₂ linked dimer, or an R₃—R*₃ linked dimer. In some embodiments, the dimer is an R₁-R*₁ linked dimer.

In certain embodiments, the two units of the Formula Ia* dimer are attached (i.e., linked) via a linker, or a linking moiety. The term “linker” as used herein refers to a chemical moiety which is attached to at least two other chemical moieties and thereby connects (“links”) those moieties. In certain embodiments the linker is preferably a low alkyl having 1-6 carbon atoms, and more preferably a methylene.

In certain embodiments, a Category I compound of Formula Ia* is:

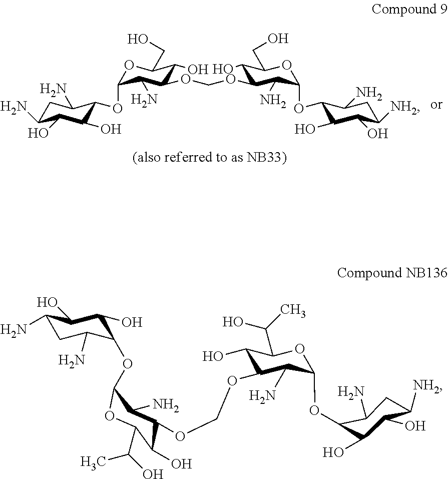

or a stereoisomer or pharmaceutically acceptable salt thereof.

### Category II Compounds

Category II compounds include all aminoglycosides disclosed or claimed in any application or patent claiming priority to U.S. Provisional Appl. No. 61/414,956, filed Nov. 18, 2010, and/or PCT Appl. No. PCT/IL2011/000889 (PCT Publ. No. WO12/66546), filed Nov. 17, 2011. These include, but are not limited to, U.S. Pat. Nos. 8,895,519, 9,175,029, 9,616,079, and 9,943,533; and EP Patent No. 2640734.

Examples of Category II compounds include, without limitation, compounds of Formula III:

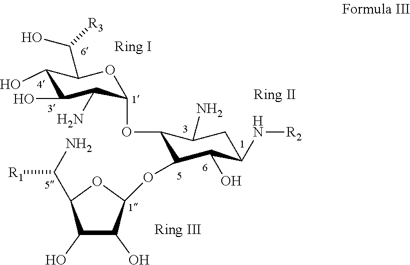

or stereoisomers or pharmaceutically acceptable salts thereof, wherein:

R₁ is selected from the group consisting of alkyl, cycloalkyl, and aryl, and is preferably alkyl;

R₂ is hydrogen or AHB;

R₃ is selected from the group consisting of hydrogen, alkyl, cycloalkyl, and aryl, and is preferably hydrogen or alkyl; and

a stereo-configuration of each of position 6′ and position 5″ is independently an R configuration or an S configuration.

In some embodiments, cycloalkyl groups include, for example, cyclopropyl, cyclobutyl, cyclopentyl, cyclohexyl, or cyclododecyl.

In certain embodiments, a Category II compound having Formula III is selected from the group consisting of:

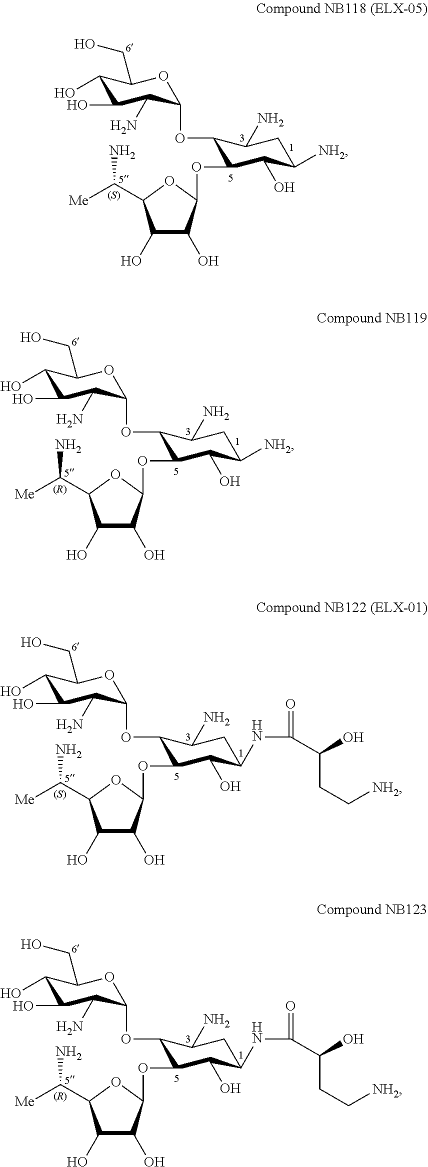

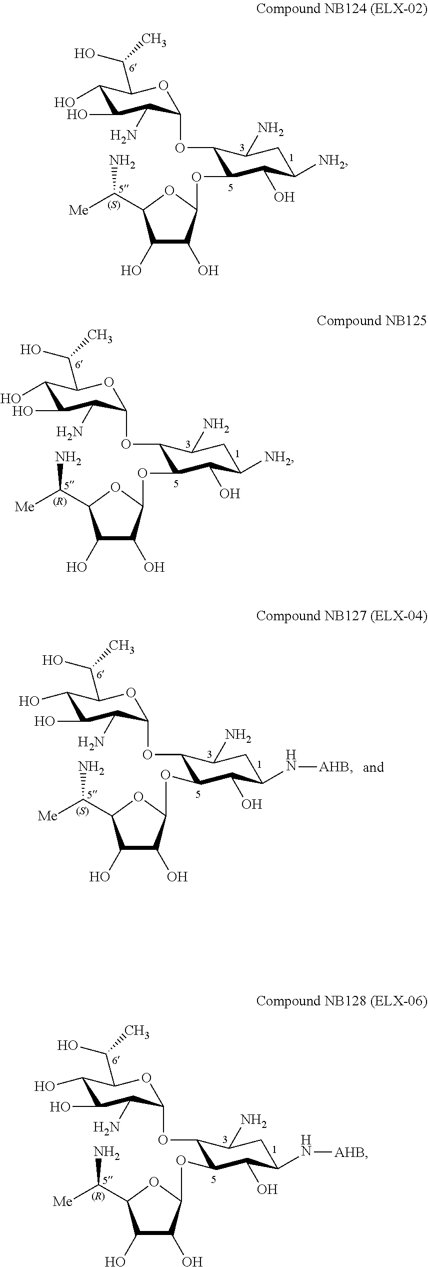

or a stereoisomer or pharmaceutically acceptable salt thereof.

### Category III Compounds

Category III compounds include all aminoglycosides disclosed or claimed in any application or patent claiming priority to one or more of US Provisional Appl. Nos. 62/213,143, filed Sep. 2, 2015; 62/213,187, filed Sep. 2, 2015; and 62/274,915, filed Jan. 5, 2016; PCT Appl. Nos. PCT/IL16/50965 (PCT Publ. No. WO17/37717), filed Sep. 2, 2016; PCT/IL/16/50966 (PCT Publ. No. WO17/37718), filed Sep. 2, 2016; PCT/IL/16/50968, filed Sep. 2, 2016 (published as WO2017/037719); and PCT/IL/16/50969 (PCT Publ. No. WO17/118698), filed Sep. 2, 2016.

Examples of Category III compounds include, without limitation, compounds of Formula IV:

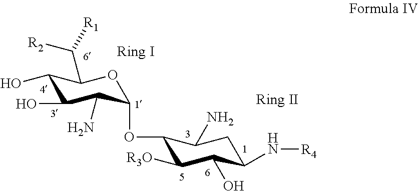

or stereoisomers or pharmaceutically acceptable salts thereof, wherein:

the dashed lines indicates a stereo-configuration of position 6′ being an R configuration or an S configuration;

R₁ is alkyl, cycloalkyl, alkyaryl, or aryl;

R₂ is selected from the group consisting of substituted or unsubstituted alkyl, OR′, and NR′R″, wherein each of R′ and R″ is independently selected from the group consisting of hydrogen, substituted or unsubstituted alkyl, substituted or unsubstituted cycloalkyl, substituted or unsubstituted aryl, substituted or unsubstituted heteroaryl, substituted or unsubstituted alkaryl, and acyl;

R₄ is selected from the group consisting of hydrogen, acyl, amino-substituted alpha-hydroxy acyl, substituted or unsubstituted alkyl, substituted or unsubstituted cycloalkyl, substituted or unsubstituted aryl, substituted or unsubstituted alkaryl, and a cell-permealizable group; and

R₃ is hydrogen, acyl, or a monosaccharide moiety represented by Formula V:

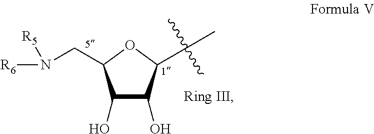

wherein:

the curved line denotes a position of attachment; and

R₅ and R₆ are each independently selected from the group consisting of hydrogen, substituted or unsubstituted alkyl, substituted or unsubstituted cycloalkyl, substituted or unsubstituted aryl, substituted or unsubstituted alkaryl, substituted or unsubstituted heteroaryl, acyl, and a cell-permealizable group or, alternatively, R₅ and R₆ together form a heterocyclic ring;

wherein when R₂ is hydroxy, R₄ is not hydrogen, AHB, or (R/S)-3-amino-2-hydroxypropionate (AHP), and/or at least one of R₅ and/or R₆, if present, is not hydrogen.

As used herein, “cell-permealizable group” refers to a group which can increase cell permeability of a compound. In some embodiments, a cell-permealizable group can include, without limitation, one or more groups selected from the group consisting of guanine, guanidyl, guanidine, hydrazinyl, hidrazide, thiohydrazide, urea, and thiourea.

In certain embodiments, a Category III compound having Formula IV is selected from the group consisting of:

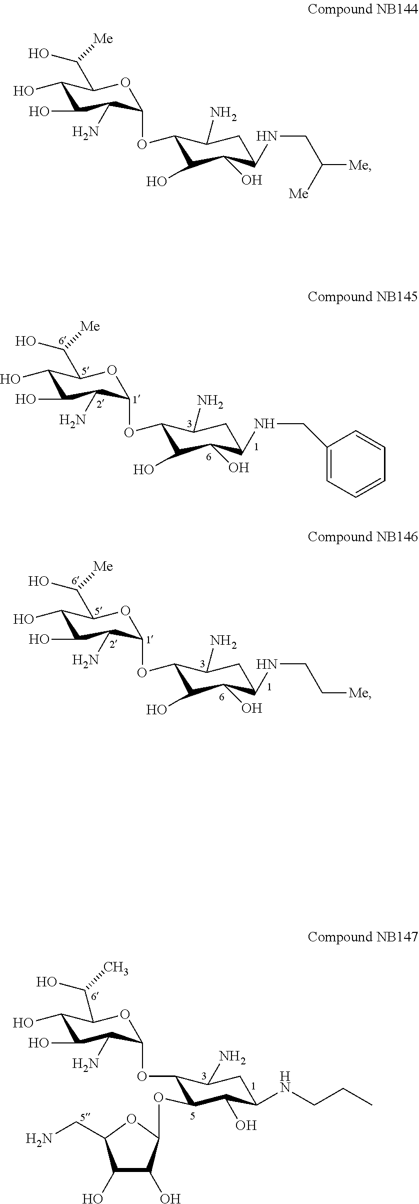

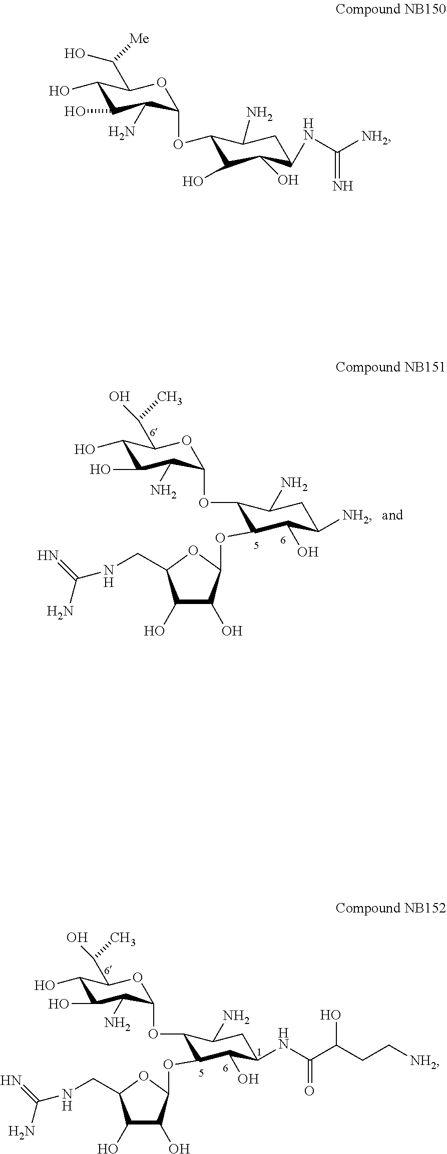

or a stereoisomer or pharmaceutically acceptable salt thereof.

In certain embodiments, the compound of Formula IV has a monosaccharide moiety at R₃, and is represented by Formula IVa:

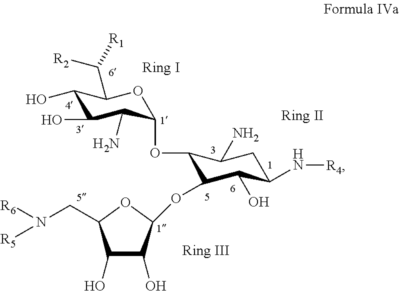

or a stereoisomer or pharmaceutically acceptable salt thereof.
wherein:

the dashed lines indicates a stereo-configuration of position 6′ being an R configuration or an S configuration;

R₁ is alkyl, cycloalkyl, alkyaryl, or aryl;

R₂ is selected from the group consisting of substituted or unsubstituted alkyl, OR′, and NR′R″, wherein each of R′ and R″ is independently selected from the group consisting of hydrogen, substituted or unsubstituted alkyl, substituted or unsubstituted cycloalkyl, substituted or unsubstituted aryl, substituted or unsubstituted heteroaryl, substituted or unsubstituted alkaryl, and acyl;

R₄ is selected from the group consisting of hydrogen, acyl, amino-substituted alpha-hydroxy acyl, substituted or unsubstituted alkyl, substituted or unsubstituted cycloalkyl, substituted or unsubstituted aryl, substituted or unsubstituted alkaryl, and a cell-permealizable group;

the curved line denotes a position of attachment; and

R₅ and R₆ are each independently selected from the group consisting of hydrogen, substituted or unsubstituted alkyl, substituted or unsubstituted cycloalkyl, substituted or unsubstituted aryl, substituted or unsubstituted alkaryl, substituted or unsubstituted heteroaryl, acyl, and a cell-permealizable group or, alternatively, R₅ and R₆ together form a heterocyclic ring;

wherein when R₂ is a hydroxyl, R₄ is not hydrogen, AHB, or (R/S)-3-amino-2-hydroxypropionate (AHP), and/or at least one of R₅ and/or R₆, if present, is not hydrogen.

In certain embodiments wherein, the compound of Formula IV has a monosaccharide moiety at R₃, and is represented by Formula IVb:

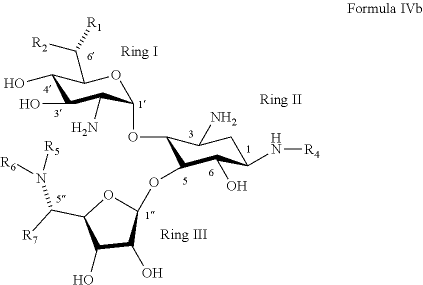

or a stereoisomer or pharmaceutically acceptable salt thereof, wherein:

the dashed lines indicate a stereo-configuration of position 6′ and/or 5″ being an R configuration or an S configuration;

R₁ is selected from the group consisting of hydrogen, alkyl, cycloalkyl, or aryl;

R₂ is selected from substituted or unsubstituted alkyl, OR′, and NR′R″, wherein each of R′ and R″ is independently selected from the group consisting of hydrogen, substituted or unsubstituted alkyl, substituted or unsubstituted cycloalkyl, substituted or unsubstituted aryl, substituted or unsubstituted heteroaryl, substituted or unsubstituted alkaryl, and acyl;

R₄ is selected from the group consisting of hydrogen, acyl, amino-substituted alpha-hydroxy acyl, substituted or unsubstituted alkyl, substituted or unsubstituted cycloalkyl, substituted or unsubstituted aryl, substituted or unsubstituted alkaryl, and a cell-permealizable group;

R₅ and R₆ are each independently selected from the group consisting of hydrogen, substituted or unsubstituted alkyl, substituted or unsubstituted cycloalkyl, substituted or unsubstituted aryl, substituted or unsubstituted alkaryl, substituted or unsubstituted heteroaryl, acyl, and a cell-permealizable group, or, alternatively, R₅ and R₆ together form a heterocyclic ring; and

R₇ is alkyl, cycloalkyl, or aryl, provided that:

when R₂ is hydroxy, R₄ is not hydrogen, AHB, or AHP, and/or at least one of R₅ and/or R₆, if present, is not hydrogen.

In certain embodiments, a Category III compound having Formula IVb is selected from the group consisting of:

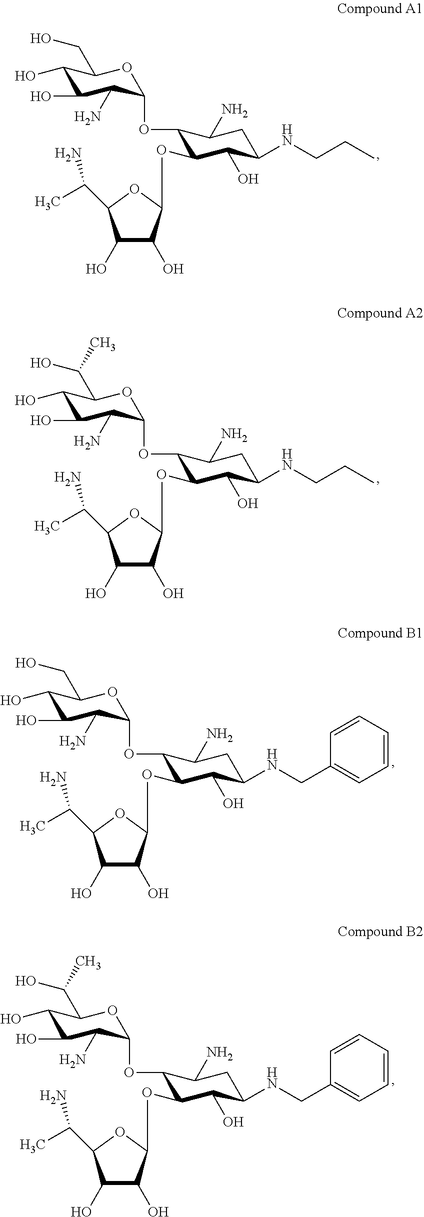

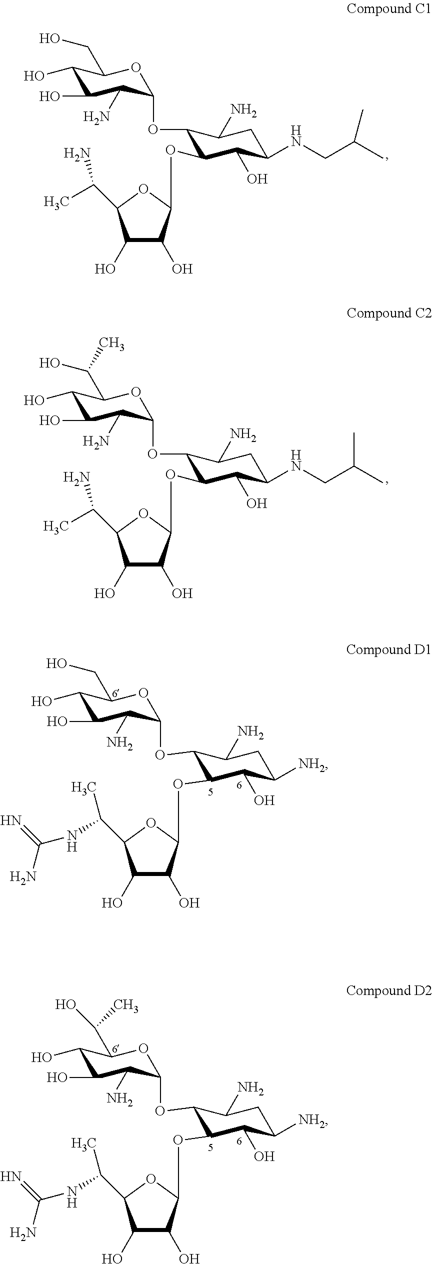

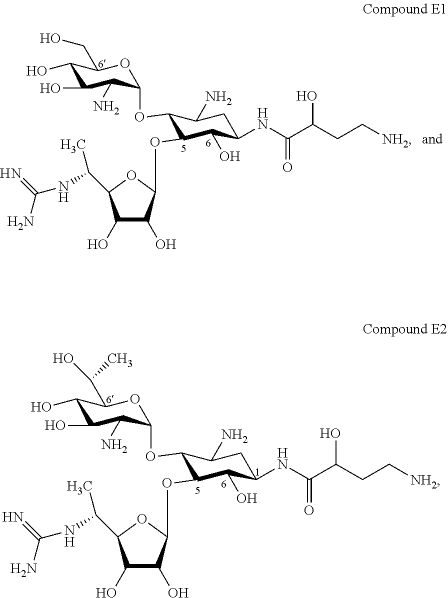

or stereoisomers or pharmaceutically acceptable salts thereof.

Additional examples of Category III compounds include, without limitation, compounds having Formula VIa:

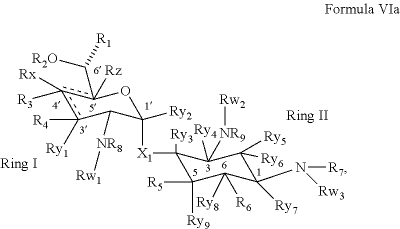

or stereoisomer or pharmaceutically acceptable salts thereof, wherein:

the dashed line indicates a stereo-configuration of position 6′ being an R configuration or an S configuration;

X₁ is O or S;

the dashed bond between C4′ and C5′ in Ring I represents a single bond or a double bond;

the dashed bond between C4′ and C3′ in Ring I represents a single bond or a double bond;

Rx, Ry₁ and Rz are each independently hydrogen, alkyl, cycloalkyl, or absent, wherein at least Rz is absent when the dashed bond between C4′ and C5′ is a double bond, and wherein at least Ry₁ is absent when the dashed bond between C4′ and C3′ is a double bond;

R₇-R₉ are each independently selected from the group consisting of hydrogen, acyl, amino-substituted alpha-hydroxy acyl, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted alkaryl, carboxylate, sulfonyl (including alkyl sulfonyl and aryl sulfonyl), and a cell-permealizable group.

Ry₂-Ry₉ and Rw₁-Rw₃ are each independently selected from the group consisting of hydrogen, alkyl, alkenyl, alkynyl, aryl, heteroaryl, and cycloalkyl, each being substituted or unsubstituted; or, alternatively, each can be hydrogen, acyl, amino-substituted alpha-hydroxy acyl, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted alkaryl, carboxylate, sulfonyl (including alkyl sulfonyl and aryl sulfonyl), or a cell-permealizable group;

R₁ is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, substituted or unsubstituted amine, substituted or unsubstituted amide, acyl, carboxylate, substituted or unsubstituted saturated hydroxyl alkyl (e.g., —CH₂—OH), and substituted or unsubstituted unsaturated hydroxy alkyl (e.g., —CH₂—OH);

R₂ is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, and acyl;

R₃ and R₄ are each independently selected from the group consisting of hydrogen, alkyl, alkenyl, alkynyl, cycloalkyl, heteroalicycle aryl, heteroaryl, amine, and OR₁₆, wherein R₁₆ is independently selected from the group consisting of hydrogen, a monosaccharide moiety, an oligosaccharide moiety, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, and acyl, or is absent, wherein R₃ is optionally absent when the dashed bond between C4′ and C5′ is a double bond, and R₄ is optionally absent when the dashed bond between C4′ and C3′ is a double bond; and

R₅ and R₆ are each independently selected from the group consisting of hydrogen, alkyl, alkenyl, alkynyl, cycloalkyl, heteroalicycle, aryl, heteroaryl, amine, and OR₁₆, wherein R₁₆ is independently selected from the group consisting of hydrogen, a monosaccharide moiety, an oligosaccharide moiety, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, and acyl.

In certain embodiments, a compound of Formula VIa comprises a monosaccharide moiety, the monosaccharide moiety comprises the structure set forth in Formula VII:

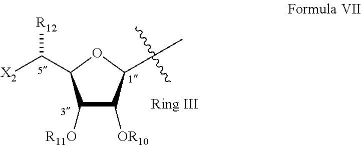

wherein:

the curved line denotes a position of attachment;

the dashed line indicates a stereo-configuration of position 5″ being an R configuration or an S configuration;

X₂ is OR₁₃ or NR₁₄R₁₅;

each of R₁₀, R₁₁ and R₁₃ is independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, and acyl;

R₁₂ is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, substituted or unsubstituted amine, substituted or unsubstituted amide, acyl, carboxylate, and saturated or unsaturated substituted hydroxyalkyl, or saturated or unsaturated unsubstituted hydroxyalkyl;

R₁₄ and R₁₅ are each independently hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, acyl, or a cell-permealizable group, or, alternatively, R₁₄ and R₁₅, when present, together form a heterocyclic ring.

Substituents not shown in Formula VII at positions such as 6′, 1″, 2″, 3″, 4″, and 5″ are independently hydrogen or substituents, such as, but not limited, as defined for Ry₂-Ry₉.

In certain embodiments, a Category III compound having Formula IVa is selected from the group consisting of:

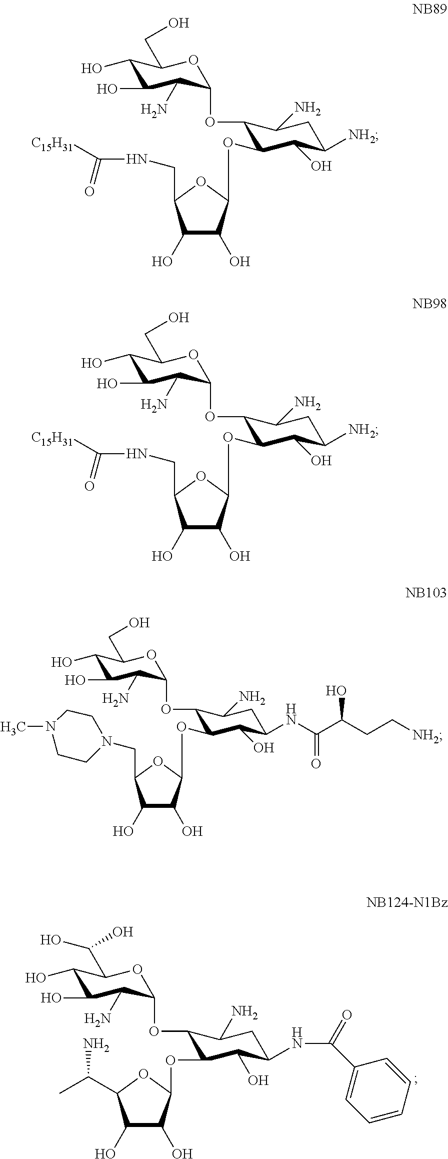

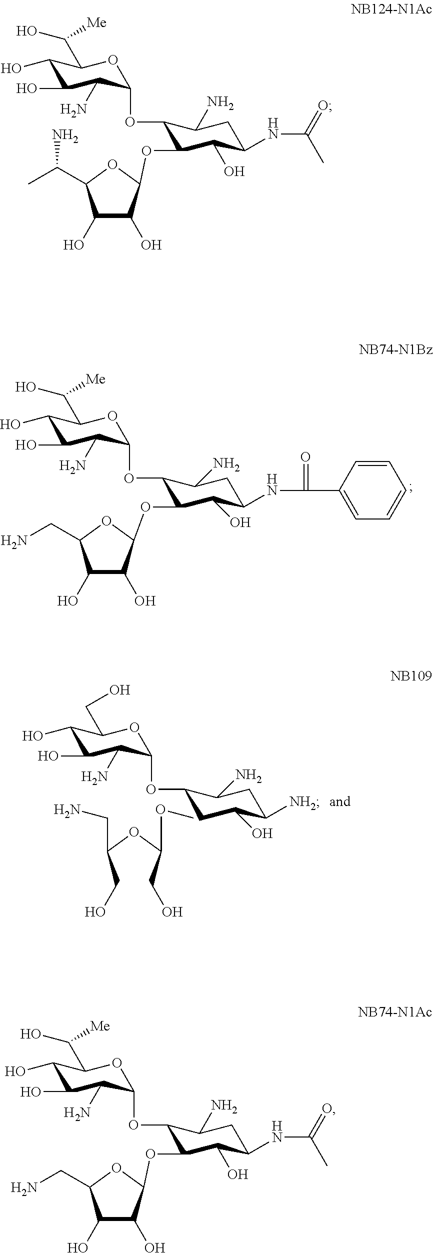

or stereoisomers or pharmaceutically acceptable salts thereof.

In certain embodiments, a compound of Formula VIa comprising a monosaccharide moiety of Formula VII has the structure set forth in Formula VIb:

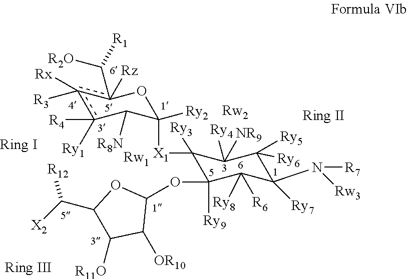

Additional examples of Category III compounds include, without limitation, compounds of Formula VIc:

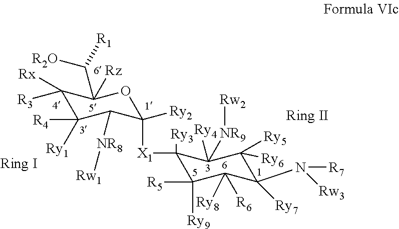

or stereoisomers or pharmaceutically acceptable salts thereof, wherein:

the dashed line indicates a stereo-configuration of position 6′ being an R configuration or an S configuration;

X₁ is O or S;

Rx, Ry₁, and Rz are each independently hydrogen, alkyl, or cycloalkyl;

R₇-R₉ are each independently selected from the group consisting of hydrogen, acyl, amino-substituted alpha-hydroxy acyl, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted alkaryl, carboxylate, sulfonyl (including alkyl sulfonyl and aryl sulfonyl), and a cell-permealizable group;

Ry₂-Ry₉ and Rw₁-Rw₃ are each independently selected from the group consisting of hydrogen, alkyl, alkenyl, alkynyl, aryl, heteroaryl, and cycloalkyl, each being substituted or unsubstituted, or, alternatively, each can be independently hydrogen, acyl, amino-substituted alpha-hydroxy acyl, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted alkaryl, carboxylate, sulfonyl (including alkyl sulfonyl and aryl sulfonyl), or a cell-permealizable group;

R₁ is a substituted or unsubstituted hydroxy alkyl (e.g., —CH₂—OH);

R₂ is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, and acyl; and

R₃ and R₄ are each independently selected from the group consisting of hydrogen, alkyl, alkenyl, alkynyl, cycloalkyl, heteroalicycle, aryl, heteroaryl, amine, and OR₁₆, wherein R₁₆ is independently selected from the group consisting of hydrogen, a monosaccharide moiety, an oligosaccharide moiety, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, and acyl, as described herein for any of the respective embodiments of Formula VIa.

Compounds represented by Formula VIc are also referred to herein as “diol-containing” compounds. In certain embodiments, diol-containing compounds are selected from the group consisting of:

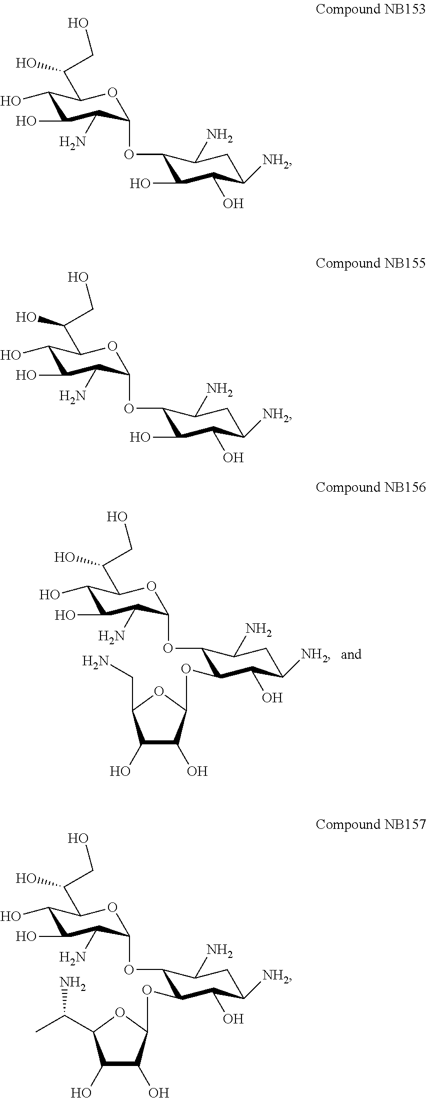

or stereoisomers or pharmaceutically acceptable salts thereof.

Additional examples of Category III compounds include, without limitation, compounds of Formula VId:

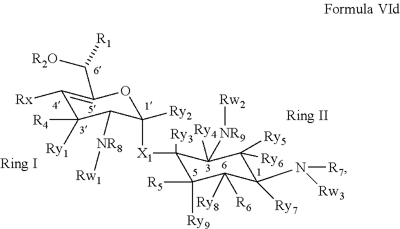

or stereoisomers or pharmaceutically acceptable salts thereof, wherein:

the dashed line indicates a stereo-configuration of position 6′ being an R configuration or an S configuration;

X₁ is O or S;

R₇-R₉ are each independently selected from the group consisting of hydrogen, acyl, amino-substituted alpha-hydroxy acyl, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted alkaryl, carboxylate, sulfonyl (including alkyl sulfonyl and aryl sulfonyl), and a cell-permealizable group;

Rx, Ry₁-Ry₉ and Rw₁-Rw₃ are each independently selected from the group consisting of hydrogen, alkyl, alkenyl, alkynyl, aryl, heteroaryl, and cycloalkyl, each being substituted or unsubstituted, or, alternatively, each can be as defined herein for R₇-R₉;

R₁ is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, substituted or unsubstituted amine, substituted or unsubstituted amide, acyl, carboxylate, substituted or unsubstituted saturated hydroxy alkyl (e.g., —CH₂—OH—), or substituted or unsubstituted unsaturated and/or substituted hydroxy alkyl, as described herein in any of the respective embodiments of Formula VIa or VIb;

R₂ is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, and acyl, as described herein in any of the respective embodiments of Formula VIa or VIb;

R₄-R₆ are each independently selected from the group consisting of hydrogen, alkyl, alkenyl, alkynyl, cycloalkyl, heteroalicycle, aryl, heteroaryl, amine, and OR₁₆, wherein R₁₆ is independently selected from hydrogen, a monosaccharide moiety, an oligosaccharide moiety, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, and acyl, as described herein in any of the respective embodiments of Formula VIa or VIb; and

Compounds represented by Formula VId are also referred to herein as “unsaturated glucosamine (Ring I)-containing” compounds. In certain embodiments, unsaturated glucosamine (Ring I)-containing compounds are selected from the group consisting of:

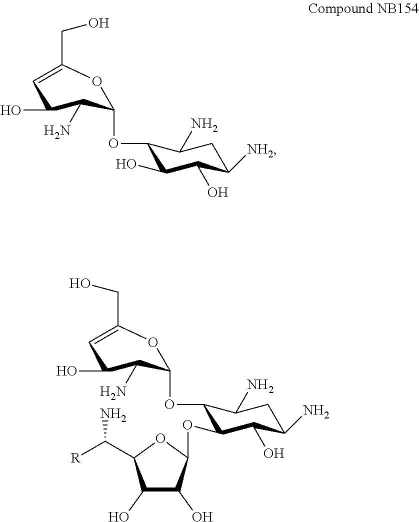

Compound NB158 (R═H), and Compound NB159 (R═CH₃),
   or stereoisomers or pharmaceutically acceptable salts thereof.

Additional examples of Category III compounds include, without limitation, compounds of Formula VIe:

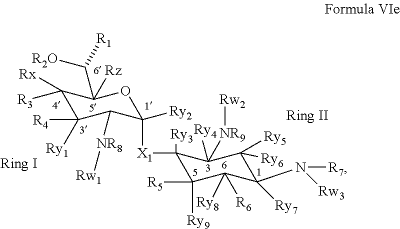

or stereoisomers or pharmaceutically acceptable salts thereof, wherein:

the dashed line indicates a stereo-configuration of position 6′ being an R configuration or an S configuration;

X₁ is O or S;

Rx, Ry₁ and Rz are each independently hydrogen, alkyl, or cycloalkyl;

R₇-R₉ are each independently selected from the group consisting of hydrogen, acyl, amino-substituted alpha-hydroxy acyl, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted alkaryl, carboxylate, sulfonyl (including alkyl sulfonyl and aryl sulfonyl), and a cell-permealizable group, as described herein for any of the respective embodiments of Formula VIa or VIb,

Ry₂-Ry₉ and Rw₁-Rw₃ are each independently selected from the group consisting of hydrogen, alkyl, alkenyl, alkynyl, aryl, heteroaryl, and cycloalkyl, each being substituted or unsubstituted, or, alternatively, each can be as defined herein for R₇-R₉;

R₁ is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, substituted or unsubstituted amine, substituted or unsubstituted amide, acyl, carboxylate, substituted or unsubstituted saturated hydroxyl alkyl (e.g., CH₂—OH), or substituted or unsubstituted unsaturated hydroxy alkyl, as described herein in any of the respective embodiments of Formula VIa or VIb;

R₂ is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, and acyl, as described herein for Formula VIa;

R₃ and R₄ are each independently selected from the group consisting of hydrogen, alkyl, alkenyl, alkynyl, cycloalkyl, heteroalicycle, aryl, heteroaryl, amine, and OR₁₆, wherein R₁₆ is independently selected from hydrogen, a monosaccharide moiety, an oligosaccharide moiety, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted alkaryl, or acyl, as described herein for any of the respective embodiments of Formula VIa;

wherein at least one of R₃-R₆ is OR₁₆, at least two of substituents R₂ and R₃-R₆ are OR₁₆, and OR₁₆ is an acyl.

Additional examples of Category III compounds include, without limitation, compounds of Formula VIII:

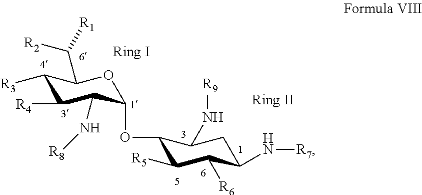

or stereoisomers a pharmaceutically acceptable salt thereof, wherein:

the dashed line indicates a stereo-configuration of position 6′ being an R configuration or an S configuration;

R₁ is selected from the group consisting of a hydroxy-substituted alkyl, a hydroxy-substituted alkenyl, a hydroxy-substituted cycloalkyl and a hydroxy-substituted aryl;

R₂ is selected from the group consisting of hydrogen, substituted or unsubstituted alkyl, and OR′, wherein R′ is selected from the group consisting of hydrogen, substituted or unsubstituted alkyl, substituted or unsubstituted cycloalkyl, substituted or unsubstituted aryl, substituted or unsubstituted heteroaryl, substituted or unsubstituted alkaryl, and acyl;

R₃-R₆ are each independently selected from the group consisting of hydrogen, substituted or unsubstituted alkyl, and OR′, wherein R′ is selected from the group consisting of hydrogen, a monosaccharide moiety, an oligosaccharide moiety, substituted or unsubstituted alkyl, substituted or unsubstituted cycloalkyl, substituted or unsubstituted aryl, substituted or unsubstituted heteroaryl, substituted or unsubstituted alkaryl, and acyl; and

R₇-R₉ are each independently selected from the group consisting of hydrogen, acyl, amino-substituted alpha-hydroxy acyl, substituted or unsubstituted alkyl, substituted or unsubstituted cycloalkyl, substituted or unsubstituted aryl, substituted or unsubstituted alkaryl, and a cell-permealizable group.

In certain embodiments, a compound of Formula VIII is selected from the group consisting of NB153 and NB155.

In certain embodiments, a compound of Formula VIII comprises a monosaccharide moiety, the monosaccharide moiety has the structure set forth in Formula IX:

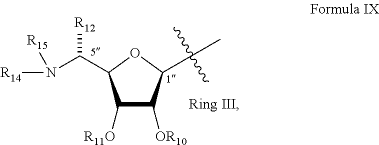

wherein:

the curved line denotes a position of attachment;

the dashed line indicates a stereo-configuration of position 5″ being an R configuration or an S configuration;

R₁₀ and R₁₁ are each independently selected from the group consisting of hydrogen, substituted or unsubstituted alkyl, substituted or unsubstituted cycloalkyl, substituted or unsubstituted aryl, substituted or unsubstituted alkaryl, and acyl;

R₁₂ is hydrogen, substituted or unsubstituted alkyl, substituted or unsubstituted cycloalkyl, or substituted or unsubstituted aryl;

each of R₁₄ and R₁₅ is independently selected from the group consisting of hydrogen, acyl, amino-substituted alpha-hydroxy acyl, substituted or unsubstituted alkyl, substituted or unsubstituted cycloalkyl, substituted or unsubstituted aryl, substituted or unsubstituted alkaryl, and a cell-permealizable group, or, alternatively, R₁₄ and R₁₅ together form a heterocyclic ring.

In certain embodiments, a compound of Formula VIII comprising a monosaccharide moiety of Formula IX has the structure set forth in Formula VIIIa:

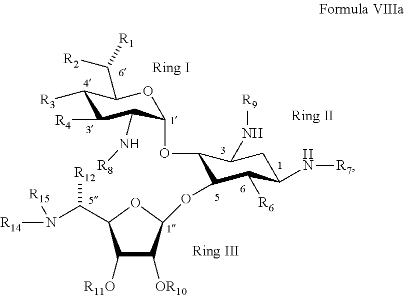

wherein:

the variables are as described herein for Formula VIII and Formula IX, including any combination thereof.

In certain embodiments, a compound of Formula VIII is selected from the group consisting of NB156 and NB157.

Additional examples of Category III compounds include, without limitation, compounds of Formula X:

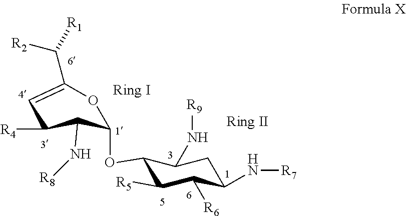

or stereoisomers or pharmaceutically acceptable salts thereof, wherein:

the dashed line indicates an optional stereo-configuration of position 6′ being R configuration or an S configuration;

R₁ is selected from the group consisting of hydrogen, substituted or unsubstituted alkyl, substituted or unsubstituted cycloalkyl, and substituted or unsubstituted aryl;

R₂ is selected from the group consisting of hydrogen, substituted or unsubstituted alkyl, and OR′, wherein R′ is selected from the group consisting of hydrogen, substituted or unsubstituted alkyl, substituted or unsubstituted cycloalkyl, substituted or unsubstituted aryl, substituted or unsubstituted heteroaryl, substituted or unsubstituted alkaryl, and acyl;

R₄-R₆ are each independently selected from the group consisting of hydrogen, substituted or unsubstituted alkyl, and OR′, wherein R′ is selected from the group consisting of hydrogen, a monosaccharide moiety, an oligosaccharide moiety, substituted or unsubstituted alkyl, substituted or unsubstituted cycloalkyl, substituted or unsubstituted aryl, substituted or unsubstituted heteroaryl, substituted or unsubstituted alkaryl, and acyl; and

R₇-R₉ are each independently selected from the group consisting of hydrogen, acyl, amino-substituted alpha-hydroxy acyl, substituted or unsubstituted alkyl, substituted or unsubstituted cycloalkyl, substituted or unsubstituted aryl, substituted or unsubstituted alkaryl, and a cell-permealizable group.

Compounds represented by Formula X are also referred to herein as “unsaturated glucosamine (Ring I)-containing” compounds.

In certain embodiments wherein, a compound of Formula X comprises a monosaccharide moiety, the monosaccharide moiety is a monosaccharide moiety of Formula IX as defined above. In certain of these embodiments, the compound of Formula X has the structure set forth in Formula Xa:

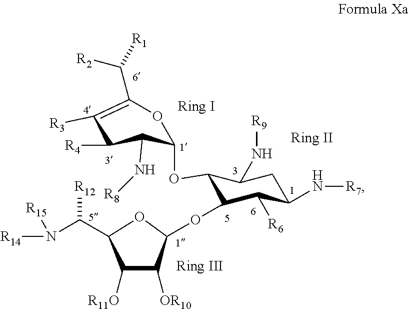

or a stereoisomer or pharmaceutically acceptable salt thereof, wherein:

each of the variables is independently as defined herein in any of the respective embodiments and any combination thereof.

In some embodiments, R₁₄ and/or R₁₅ are alkyl substituted with a carbonyl or oxo (i.e., C═O) group (e.g., —CO(C₁₅H₃₁)). In some embodiments, R₁₄ and R₁₅ form together a nitrogen-containing heterocyclic ring, such as, but not limited to, morpholine, piperidine, and piperazine. In some embodiments, R₁₅ and/or R₁₄ are a guanidine group (guanidinyl; guanidyl). In some embodiments, R₁₅ and/or R₁₄ is independently an alkyl, a cell-permealizable group, as described herein, or an acyl, such as, for example, an alpha-hydroxy acyl or an amino-substituted alpha-hydroxy acyl, as described herein. R₁₄ and R₁₅, if present, include, but are not limited to, hydrogen, (R/S)-4-amino-2-hydroxybutyryl (AHB), (R/S)-3-amino-2-hydroxypropionyl (AHP), 5-aminopentanoyl, 5-hydroxypentanoyl, formyl, —C(=0)-0-methyl, —C(=0)-0-ethyl, —C(=0)-0-benzyl, —β-amino-a-hydroxypropionyl, —δ-amino-a-hydroxyvaleryl, -β-benzyloxycarbonylamino-a-hydroxypropionyl, —δ-benzyloxycarbonylamino-a-hydroxyvaleryl, methylsulfonyl, phenylsulfonyl, benzoyl, propyl, isopropyl, —(CH₂)₂NH₂, —(CH)₃NH₂, —CH₂CH(NH₂)CH₃, —(CH₄NH₂, —(CH₂)₅NH₂, —(CH₂)₂NH-ethyl, —(CH₂)₂NH(CH₂)₂NH₂, —(CH₂)₃NH(CH₂)₃NH₂, —(CH₂)₃NH(CH₂)₄NH(CH₂)₃NH₂, —CH(—NH₂)CH₂(OH), —CH(—OH)CH₂(NH₂), —CH(—OH)—(CH₂)₂(NH₂), —CH(-NH₂)—(CH₂)₂(OH), —CH(—CH₂NH₂)—(CH₂OH), —(CH₂)₄NH(CH₂)₃NH₂, —(CH₂)₂NH(CH₂)₂NH(CH₂)₂NH₂, —(CH₂)₂N(CH₂CH₂NH₂)₂, —CH₂-C(=0)NH₂, —CH(CH₃)—C(=0)NH₂, —CH₂-phenyl, —CH(i-propyl)-C(=0)NH₂, —CH(benzyl)-C(=0)NH₂, —(CH₂)₂OH, —(CH₂)₃OH and —CH(CH₂OH)₂.

In some embodiments, R₇ is selected from the group consisting of hydrogen, (R/S)-4-amino-2-hydroxybutyryl (AHB), (R/S)-3-amino-2-hydroxypropionate (AHP), and (R/S)-3-amino-2-hydroxypropionyl, 5-aminopentanoyl, 5-hydroxypentanoyl, formyl, —C(═O)—O-methyl, —C(═O)—O-ethyl, —C(═O)—O-benzyl, -β-amino-a-hydroxypropionyl, -δ-amino-a-hydroxyvaleryl, —β-benzyloxycarbonylamino-a-hydroxypropionyl, —δ— benzyloxycarbonylamino-a-hydroxyvaleryl, methylsulfonyl, phenylsulfonyl, benzoyl, propyl, isopropyl, —(CH₂)₂NH₂, —(CH)₃NH₂, —CH₂CH(NH₂)CH3, —(CH)₄NH₂, —(CH₂)₅NH₂, —(CH₂)₂NH-ethyl, —(CH₂)₂NH(CH₂)₂NH₂, —(CH₂)₃NH(CH₂)₃NH₂, —(CH₂)₃NH(CH₂)₄NH(CH₂)₃NH₂, —CH(—NH₂)CH₂(OH), —CH(—OH)CH₂(NH₂), —CH(—OH)—(CH₂)₂(NH₂), —CH(-NH₂)—(CH₂)₂(OH), —CH(—CH₂NH₂)—(CH₂OH), —(CH₂)₄NH(CH₂)₃NH₂, —(CH₂)NH(CH₂)NH(CH₂)₂NH₂, —(CH₂)₂N(CH₂CH₂NH₂)₂, —CH₂—C(═O)NH₂, —CH(CH₃)—C(═O)NH₂, —CH₂-phenyl, —CH(i-propyl)-C(═O)NH₂, —CH(benzyl)-C(═O)NH₂, —(CH₂)₂OH, —(CH₂)₃OH and —CH(CH₂OH)₂. In some embodiments, R₇ is alkyl, cycloalkyl or aryl. In some embodiments, R₇ is an alkyl selected from the group consisting of ethyl, propyl, isopropyl, isobutyl, tert-butyl, and benzyl. In some embodiments, R₇ is hydrogen, acyl or amino-substituted a-hydroxy-acyl. In some embodiments, R₇ is a substituted and/or unsubstituted methyl or ethyl. In some of these embodiments, the methyl or ethyl is substituted by, for example, a cycloalkyl or aryl In some embodiments, R₇ is cycloalkyl, and the cycloalkyl can be, for example, cyclopropyl, cyclobutyl, cyclopentyl and cyclohexyl. In some embodiments, R₇ is aryl, and the aryl can be, for example, a substituted or unsubstituted phenyl. Non-limiting examples include unsubstituted phenyl and toluene. In some embodiments, R⁷ is an aryl acyl (e.g., —C(═O)—R′, wherein R′ is an aryl, e.g, a benzene).

### Category IV Compounds

Category IV compounds include all aminoglycosides disclosed in the applications and patents claiming priority to U.S. Provisional Appl. No. 62/515,021, filed Jun. 5, 2017, and/or PCT Appl. No. PCT/IL18/50612, filed Jun. 5, 2018.

Examples of Category IV compounds include, without limitation, compounds of Formula XI:

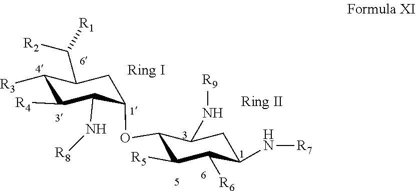

or stereoisomers or pharmaceutically acceptable salts thereof, wherein:

the dashed line indicates a stereo-configuration of position 6′ being an R configuration or an S configuration;

R₁ is selected from the group consisting of hydrogen, substituted or unsubstituted alkyl, substituted or unsubstituted cycloalkyl, and substituted or unsubstituted aryl;

R₂ is selected from the group consisting of hydrogen, substituted or unsubstituted alkyl, and ORx, wherein Rx is selected from the group consisting of hydrogen, substituted or unsubstituted alkyl, substituted or unsubstituted cycloalkyl, substituted or unsubstituted aryl, substituted or unsubstituted heteroaryl, substituted or unsubstituted alkaryl, or an acyl, or alternatively R₂ is ORx and together with R₃ forms a dioxane;

R₃ is selected from the group consisting of hydrogen, substituted or unsubstituted alkyl, and ORy, wherein Ry is selected from the group consisting of hydrogen, substituted or unsubstituted alkyl, substituted or unsubstituted cycloalkyl, substituted or unsubstituted aryl, substituted or unsubstituted heteroaryl, substituted or unsubstituted alkaryl, and acyl, or, alternatively, R₃ is ORy and together with R₂ forms a dioxane;

R₄-R₆ are each independently selected from the group consisting of hydrogen, substituted or unsubstituted alkyl, and ORz, wherein Rz is selected from the group consisting of hydrogen, a monosaccharide moiety, an oligosaccharide moiety, substituted or unsubstituted alkyl, substituted or unsubstituted cycloalkyl, substituted or unsubstituted aryl, substituted or unsubstituted heteroaryl, substituted or unsubstituted alkaryl, and acyl; and

R₇-R₉ are each independently selected from the group consisting of hydrogen, acyl, amino-substituted alpha-hydroxy acyl, substituted or unsubstituted alkyl, substituted or unsubstituted cycloalkyl, substituted or unsubstituted aryl, substituted or unsubstituted alkaryl, and sulfonyl,

provided that at least one of R₇-R₉ is a sulfonyl.

In certain embodiments, a compound of Formula XI in which R₇ is sulfonyl has the structure of Formula XIa:

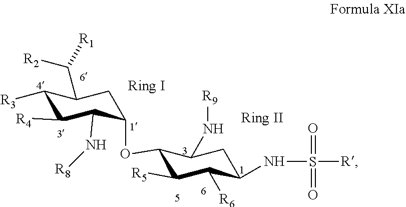

or a stereoisomer or pharmaceutically acceptable salt thereof, wherein:

R₆, R₈, and R₉ are as defined for Formula XI; and

R′ is selected from the group consisting of hydrogen, substituted or unsubstituted alkyl, substituted or unsubstituted cycloalkyl, substituted or unsubstituted alkaryl, and substituted or unsubstituted aryl.

In certain embodiments, a compound of Formula XI is a compound of Formula XI*:

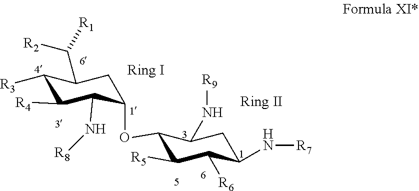

or a stereoisomer or pharmaceutically acceptable salt thereof, wherein:

the dashed line indicates a stereo-configuration of position 6′ being an R configuration or an S configuration;

R₁ is selected from the group consisting of hydrogen, substituted or unsubstituted alkyl, substituted or unsubstituted cycloalkyl, and substituted or unsubstituted aryl;

R₂ is ORx, wherein Rx is selected from the group consisting of substituted or unsubstituted alkyl, substituted or unsubstituted cycloalkyl, substituted or unsubstituted aryl, substituted or unsubstituted heteroaryl, and substituted or unsubstituted alkaryl;

R₃ is ORy, wherein Ry is selected from the group consisting of substituted or unsubstituted alkyl, substituted or unsubstituted cycloalkyl, substituted or unsubstituted aryl, substituted or unsubstituted heteroaryl, and substituted or unsubstituted alkaryl;

R₄-R₆ are each independently selected from the group consisting of hydrogen, substituted or unsubstituted alkyl, and ORz, wherein Rz is selected from the group consisting of hydrogen, a monosaccharide moiety, an oligosaccharide moiety, substituted or unsubstituted alkyl, substituted or unsubstituted cycloalkyl, substituted or unsubstituted aryl, substituted or unsubstituted heteroaryl, substituted or unsubstituted alkaryl, and acyl; and

R₇-R₉ are each independently selected from the group consisting of hydrogen, acyl, amino-substituted alpha-hydroxy acyl, substituted or unsubstituted alkyl, substituted or unsubstituted cycloalkyl, substituted or unsubstituted aryl, substituted or unsubstituted alkaryl, and sulfonyl, and wherein ORx and ORy are linked to one another such that R₂ and R₃ together form a dioxane.

In certain embodiments, a compound of Formula XI is a compound of Formula XI*a:

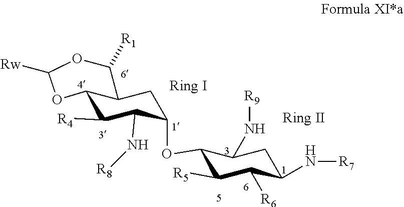

or a stereoisomer or pharmaceutically acceptable salt thereof, wherein:

R₁, R₄-R₆ and R₇-R₉ are as defined for Formula XI*; and

Rw is selected from the group consisting of hydrogen, substituted or unsubstituted alkyl, substituted or unsubstituted cycloalkyl, substituted or unsubstituted alkaryl, and substituted or unsubstituted aryl.

In certain embodiments, a compound of Formula XI is a compound of Formula XI*b:

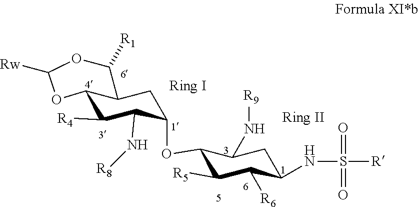

or a stereoisomer or pharmaceutically acceptable salt thereof, wherein:

Rw, R₁, R₄-R₆, R₈, and R₉ are as defined for Formula XI*; and

R′ is selected from the group consisting of hydrogen, substituted or unsubstituted alkyl, substituted or unsubstituted cycloalkyl, substituted or unsubstituted alkaryl, and substituted or unsubstituted aryl.

In certain embodiments wherein, the compound of Formula XI comprises a monosaccharide moiety, the monosaccharide moiety has the structure set forth in Formula IX. In certain of these embodiments, the compound has the structure of Formula XII:

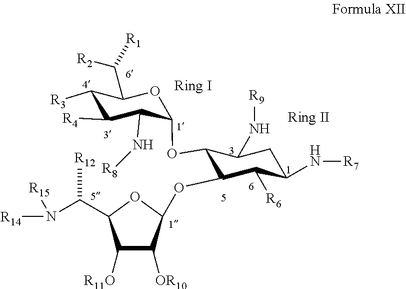

or a stereoisomer or pharmaceutically acceptable salt thereof, wherein:

R₁-R₄ and R₆-R₉ are each as defined for Formula X or Formula Xa; and

R₁₀, R₁₁, R₁₂, R₁₄, and R₁₅ are each as defined for Formula IX.

In certain embodiments of the compound of Formula XII wherein R₇ is sulfonyl, the compound has the structure of Formula XIIa:

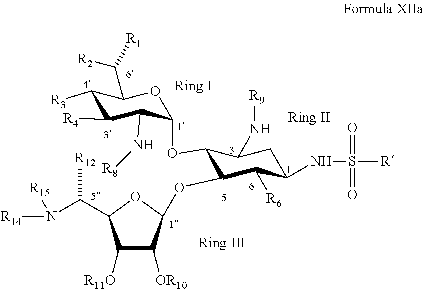

or a stereoisomer or pharmaceutically acceptable salt thereof, wherein:

R₁-R₄, R₆, R₈, and R₉ are as defined for Formula X or Formula Xa;

R₁₀, R₁₁, R₁₂, R₁₄, and R₁₅ are as defined for Formula IX; and

R′ is selected from the group consisting of hydrogen, substituted or unsubstituted alkyl, substituted or unsubstituted cycloalkyl, substituted or unsubstituted alkaryl, and substituted or unsubstituted aryl.

In certain embodiments, a compound of Formula XIIa is selected from the group consisting of:

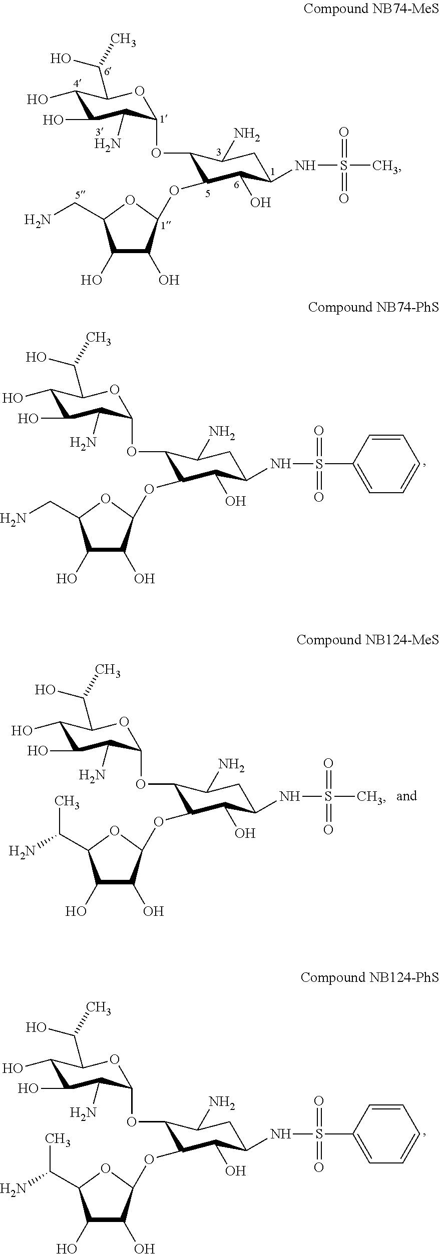

and stereoisomer or pharmaceutically acceptable salts thereof.

In certain embodiments of the compound of Formula XII wherein R₅ is ORz and Rz is the monosaccharide moiety represented by Formula IX, the compound has the structure of Formula XII*:

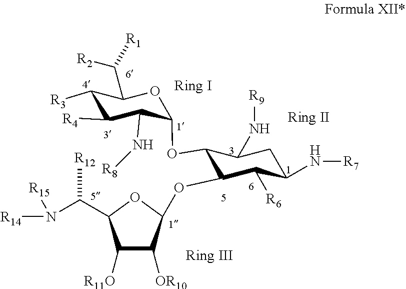

or is a stereoisomer or pharmaceutically acceptable salt thereof, wherein:

R₁-R₄ and R₆-R₉ are each as defined for Formula X* or X*a or X*b; and

R₁₀, R₁₁, R₁₂, R₁₄, and R₁₅ are each as defined for Formula XI.

In certain embodiments of the compound of Formula XII wherein the compound comprises a dioxane and the dioxane is a substituted or unsubstituted 1,3-dioxane, the compound has the structure of Formula XII*a:

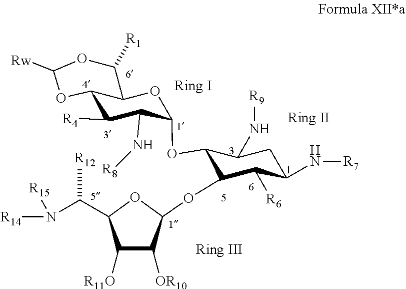

or is a stereoisomer or pharmaceutically acceptable salt thereof, wherein:

R₁, R₄, R₆, and R₇-R₉ are as defined for Formula X* or X*a or X*b;

R₁₀, R₁₁, R₁₂, R₁₄, and R₁₅ are as defined for Formula XI; and

Rw is selected from the group consisting of hydrogen, substituted or unsubstituted alkyl, substituted or unsubstituted cycloalkyl, substituted or unsubstituted alkaryl, and substituted or unsubstituted aryl.

In other embodiments wherein, the compound of Formula XII comprises a dioxane and the dioxane is a substituted or unsubstituted 1,3-dioxane, the compound has the structure of Formula XII*b:

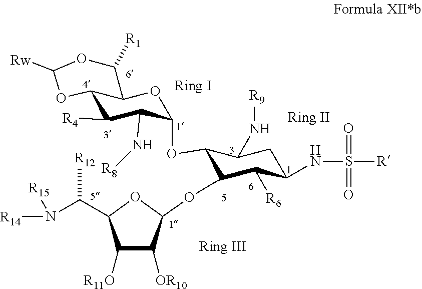

or is a stereoisomer or pharmaceutically acceptable salt thereof, wherein:

Rw, R₁, R₄, R₆, R₈, and R₉ are as defined for Formula X*b;

R₁₀, R₁₁, R₁₂, R₁₄, and R₁₅ are as defined for Formula IX; and

R′ is selected from the group consisting of hydrogen, substituted or unsubstituted alkyl, substituted or unsubstituted cycloalkyl, substituted or unsubstituted alkaryl, and substituted or unsubstituted aryl.

Additional examples of Category IV compounds include, without limitation, compounds of Formula XIII:

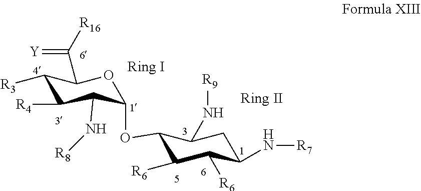

or stereoisomers or pharmaceutically acceptable salts thereof, wherein:

Y is oxygen or sulfur;

R₁₆ is selected from the group consisting of hydrogen, amine, and ORq;

Rq is selected from the group consisting of hydrogen, a monosaccharide moiety, an oligosaccharide moiety, substituted or unsubstituted alkyl, substituted or unsubstituted cycloalkyl, substituted or unsubstituted aryl, substituted or unsubstituted heteroaryl, and substituted or unsubstituted alkaryl;

R₃-R₆ are each independently selected from the group consisting of hydrogen, substituted or unsubstituted aryl, unsubstituted alkyl, and ORz, wherein Rz is selected from hydrogen, a monosaccharide moiety, an oligosaccharide moiety, substituted or unsubstituted alkyl, substituted or unsubstituted cycloalkyl, substituted or unsubstituted aryl, substituted or unsubstituted heteroaryl, substituted or unsubstituted alkaryl, and acyl; and

R₇-R₉ are each independently selected from the group consisting of hydrogen, acyl, amino-substituted alpha-hydroxy acyl, substituted or unsubstituted alkyl, substituted or unsubstituted cycloalkyl, substituted or unsubstituted aryl, substituted or unsubstituted alkaryl, and sulfonyl.

In certain embodiments, a compound of Formula XIII is selected from the group consisting of:

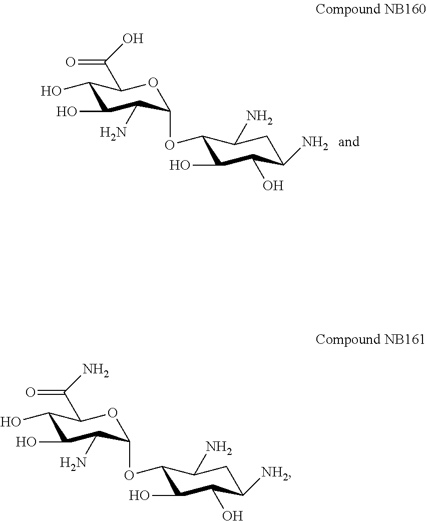

or stereoisomers or pharmaceutically acceptable salts thereof.

In certain embodiments of the compound of Formula XIII, wherein R₅ is ORz, and Rz is the monosaccharide moiety represented by Formula XI, the compound has the structure of Formula XIIIa:

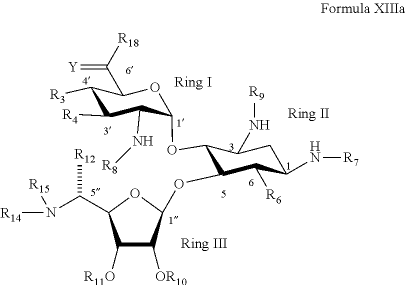

or a stereoisomer or pharmaceutically acceptable salt thereof, wherein:

the dashed line indicates a stereo-configuration of position 5″ being each independently an R configuration or an S configuration;

Y, R₃, R₄, and R₆-R₉ are each as defined for Formula XIII;

R₁₀ and R₁₁ are each independently selected from the group consisting of hydrogen, substituted or unsubstituted alkyl, substituted or unsubstituted cycloalkyl, substituted or unsubstituted aryl, substituted or unsubstituted alkaryl, and acyl;

R₁₂ is selected from the group consisting of hydrogen, substituted or unsubstituted alkyl, substituted or unsubstituted cycloalkyl, and substituted or unsubstituted aryl; and

each of R₁₄ and R₁₅ is independently selected from the group consisting of hydrogen, acyl, amino-substituted alpha-hydroxy acyl, substituted or unsubstituted alkyl, substituted or unsubstituted cycloalkyl, substituted or unsubstituted aryl, substituted or unsubstituted alkaryl, sulfonyl, and a cell-permealizable group, or, alternatively, R₁₄ and R₁₅ together form a heterocyclic ring.

In certain embodiments, a compound of Formula XIIIa is selected from the group consisting of:

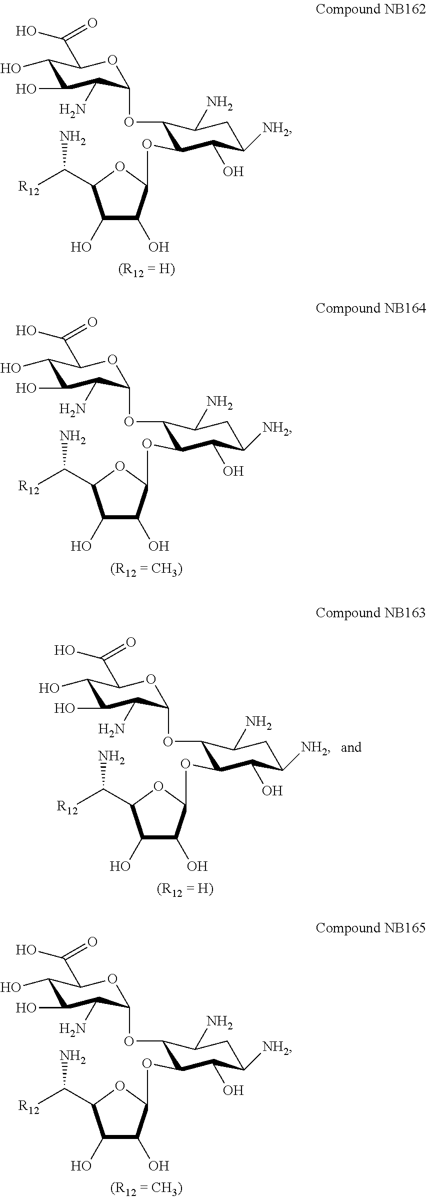

or stereoisomers or pharmaceutically acceptable salts thereof.

### Administration Routes

One or more aminoglycosides and/or derivatives thereof, or pharmaceutical formulations thereof, may be delivered to a subject by any suitable administration pathway known in the art, including but not limited to intravitreal injection (IVI) and topical administration.

Intravitreal injection is an approved mode of administration for retinal medications (e.g., gentamicin). Intravitreal pharmacokinetic data in human shows a half-life of about 40-60 hours for gentamicin. There is a limited tolerability on different aminoglycosides administered intravitreally. Aminoglycosides and/or derivatives thereof uptake to the retinal tissues is limited after systemic dose. Ocular delivery can avoid safety concerns associated with systemic exposure. Read through activities and physical properties of the aminoglycosides and/or derivatives thereof disclosed herein can achieve therapeutic effects via ocular injection. In certain embodiments, Usher syndrome can be treated by intravitreal injection.

Topical administration is another common route for administering medicinal agents to the eye. Aminoglycosides such as Tobramycin are given by this route. Any suitable formulation such as a solution or ointment that is stable at room temperature can be used. In certain embodiments, aniridia can be treated by topical administration.

### Kits

Provided herein in certain embodiments are kits for carrying out the methods disclosed herein. In certain embodiments, the kits comprise one or more aminoglycosides and/or derivatives thereof, or one or more pharmaceutical formulations comprising such aminoglycosides and/or derivatives thereof. In certain embodiments, the kits further comprise one or more additional therapeutic agents or pharmaceutical formulations thereof. In those embodiments wherein the kits comprise two or more compounds, e.g., two or more aminoglycosides and/or derivatives thereof or an aminoglycoside or derivative thereof and an additional therapeutic agent, the two or more compounds may be present in the kit in a single composition or in separate compositions. In certain embodiments, the kits comprise instructions in a tangible medium.

The foregoing and the following working examples are merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### EXAMPLES

### Example 1: Biocompatibility and Pharmacokinetic/Pharmacodynamic Analysis

Biocompatibility and pharmacokinetic/pharmacodynamic profiles were evaluated for the aminoglycosides NB30, NB54/Compound 37, NB84/ELX-03, NB122/ELX-01, NB124/ELX-02, NB127/ELX-04, NB118/ELX-05, and NB128/ELX-06 in patient-derived cell lines and rodent animal models. Overall, these studies showed that each of the tested aminoglycosides had an increased tolerability profile as compared to gentamicin and G418, which have been reported to have dose-limiting retinal toxicities, and that intravitreal injection produced retinal exposure equal or superior to the read-through threshold without the impacts on staining and retinal structure that are observed with gentamicin and G418.

To determine ocular safety parameters, an endotoxin (EU) test was performed and a retinal exposure threshold of <0.2 EU/mL was established.

The cytotoxicity of NB30, NB54, NB84, NB122, NB124, and NB127 was evaluated in retinal cultures. The staining profiles of NB30 and NB54 did not significantly differ versus the untreated retinal cultures, suggesting a favorable cytotoxicity profile for NB30 and NB54.

In furtherance of the cytotoxicity studies, NB84, NB118, and NB122 were tested for their in vivo ocular tolerance, as measured by ERG and histopathology, at intravitreal doses up to 600 μg/mL, a value far superior to gentamicin.

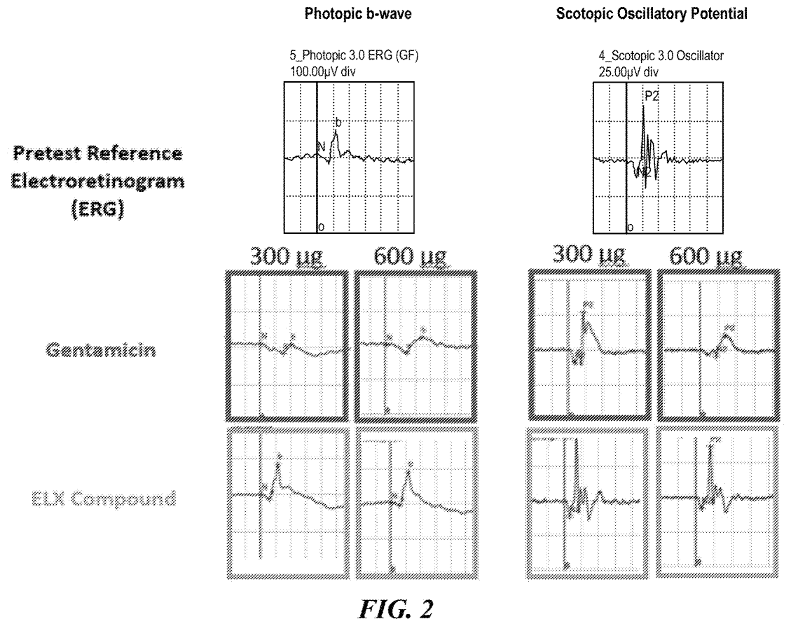

Biocompatibility and pharmacokinetic/pharmacodynamic profiles were then evaluated for intravitreal injection of NB118 versus gentamicin. Single-dose tolerability was screened in healthy New Zealand white rabbits and evaluated based on ERG and histology. Dark-adapted oscillatory potential and photopic b-wave amplitudes were measured pre- and nine-days post a single 300 μg or 600 μg injection of aminoglycoside as shown in FIG. 2. Injection with gentamicin resulted in ablation of oscillatory potential peaks and moderate reduction of b-wave potentials, while injection of NB118 demonstrated a preserved ERG (data are representative of group mean). As depicted in FIG. 2, there was minimal to no impact on amplitudes or implicit times, suggesting that there were no adverse functional changes of the retina following intravitreal administration of NB118.

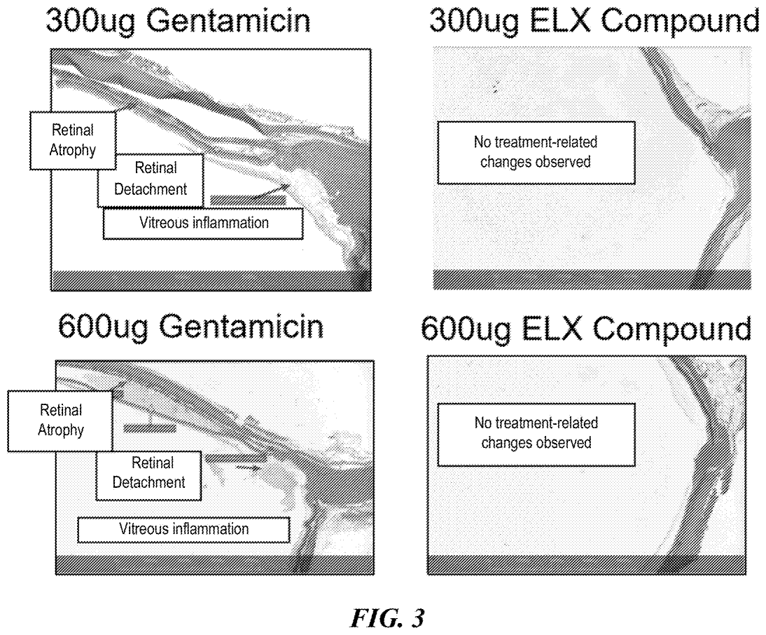

FIG. 3 depicts hematoxylin and eosin (H&E) stained sections of rabbit retinal tissue showing that intravitreal administration of NB118 did not produce adverse anatomic changes in the rabbits' retina, and that the retinal structure remained intact. Results were confirmed by a veterinary pathologist.

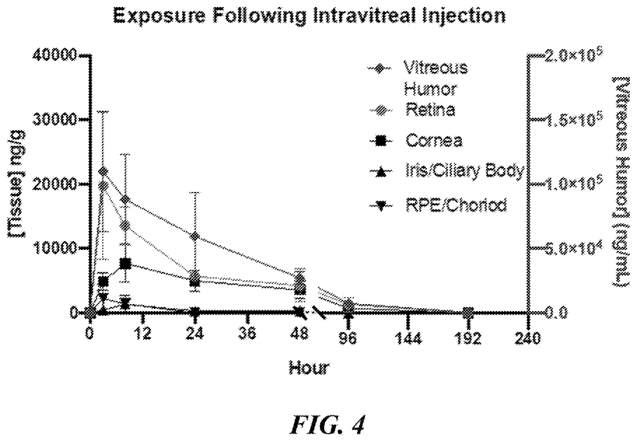

Pharmacokinetic profiles of NB124 were evaluated using healthy Dutch-Belted rabbits. Rabbits were injected with 100 μg of NB124 and followed for 192 hours. As shown in FIG. 4, peak retina exposure was observed at 3 hours and was found to cover the compounds half maximal inhibitory concentration (IC50) for over 48 hours. Overall, the results support a favorable therapeutic index for intravitreal administration of any of NB124.

NB122 was well-tolerated compared to gentamicin in gross ophthalmological examination and ERG when administered by intravitreal injection.

### Example 2: Read-Through Activity on Nonsense Mutations Associated with Usher Syndrome

The ability of NB122/ELX-01, NB84/ELX-03, NB127/ELX-04, NB118/ELX-05, NB128/ELX-06, NB124-MeS/ELX-07, and NB157/ELX-10 to induce read-through of nonsense mutations associated with Usher Syndrome was evaluated. Overall, these studies showed that the tested aminoglycosides suppress nonsense mutations associated with Usher Syndrome in both cell-free assays and in vitro cellular models, and that these effects extend across different Usher-associated gene mutations.

Usher Syndrome is associated with numerous nonsense mutations, including but not limited to mutations associated with the indications USH1 and USH2.

TABLE 1
Nonsense mutations associated with Usher syndrome

| Indication | Gene | Gene Frequency | NS Frequency |
|---|---|---|---|
| USH1B | MYO7A | ~60% | ~35% |
| USH1D | CDH23 | ~15% | ~15% |
| USH1F | PCDH15 | ~10% | ~40% |
| USH2A | USH2A | ~80% | ~35% |
| USH2C | GPR98 | ~5% | ~35% |

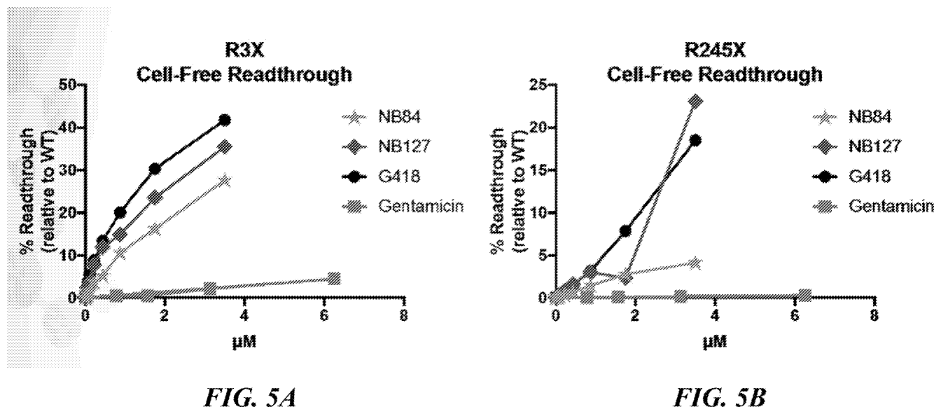

Cell-Free Read-through Assay. NB84 and NB127 were evaluated for their ability to induce read-through of the USH1F nonsense mutations R3X and R₂₄₅X in a cell-free assay. As shown in FIG. 5A-B, both compounds induced significant read-through as compared to G418 and gentamicin.

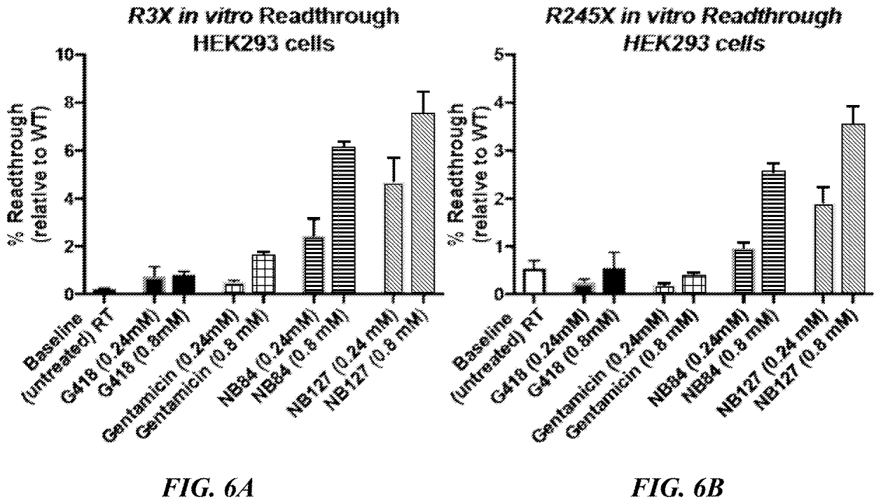

In Vitro Cell-Based Assay. NB84 and NB127 were next tested in an in vitro assay using HEK293 cells to determine if similar read-through activity could be achieved in a cellular environment. The in vitro assay showed that both compounds induced significant read-through in cells, with a greater degree of read-through at higher concentrations (FIG. 6A-B).

An in vitro assay was then performed to determine if the same dose-dependent suppression could be achieved using other aminoglycosides. This in vitro assay utilized a plasmid-based, dual-luciferase approach. Short sequences that include Usher syndrome nonsense mutations were cloned into the linker region between Renilla and Firefly luciferase reporters, and plasmids were transiently transfected into HeLa cells to evaluate compound read-through. This model permits an early assessment of read-through potential against disease-specific mutations. Read-through activity was evaluated using USH2A and USH1F mutations.

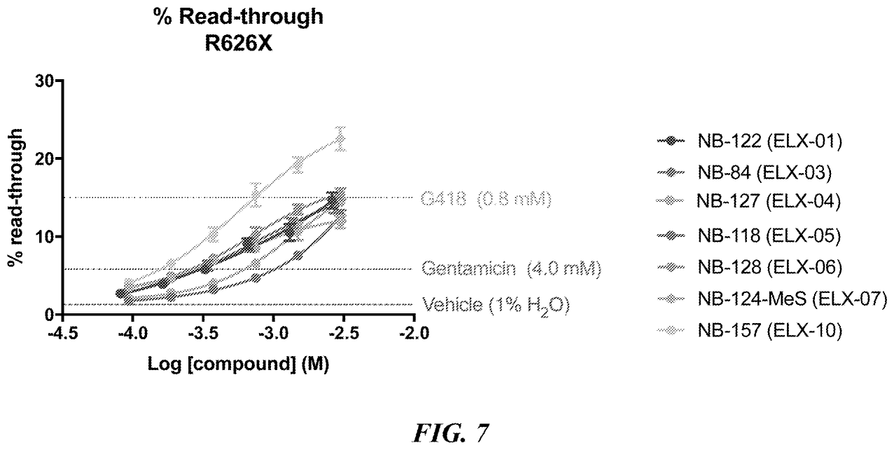

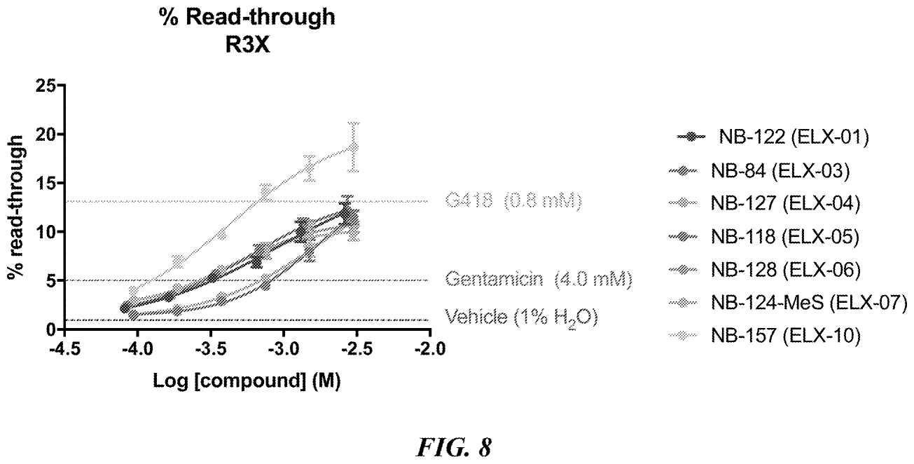

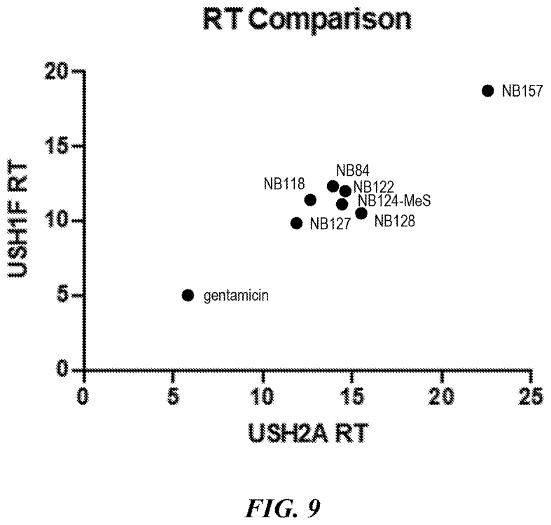

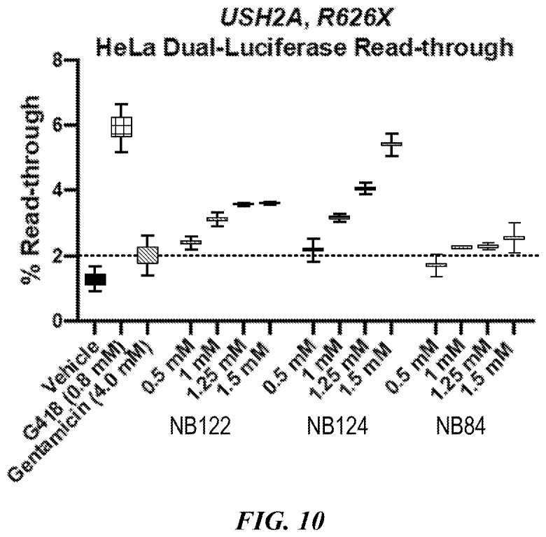

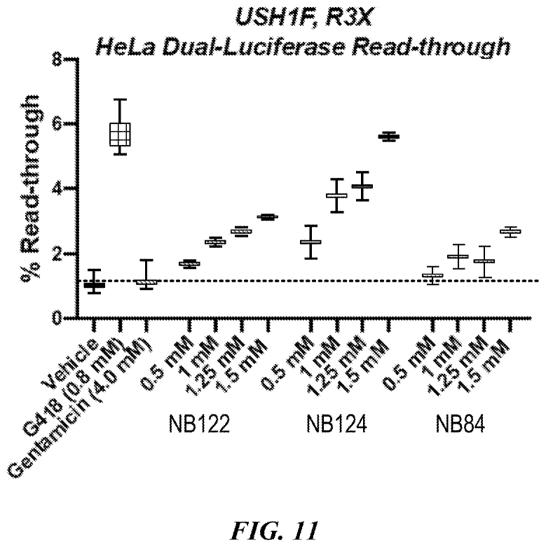

NB122, NB124, NB84, NB127, NB118, NB128, NB124-MeS, and NB157 were evaluated for their ability to induce read-through on the USH2A mutation R626X and the USH1F PCDH15 mutation R3X. NB84, NB127, NB118, NB128, NB124-MeS, and NB157 each induced significant read-through of the USH2A and USH1F mutations (FIG. 7 and FIG. 8, respectively). Comparative studies showed the percent read-through for each of the aminoglycosides was significantly higher than that of gentamicin (FIG. 9 and Table 2). Further, read-through induction by NB122, NB124, NB84, NB118, and NB128 exhibited dose-dependence (FIGS. 10 and 11, respectively). Administration of NB122 resulted in a 2.5-fold increase over native read-through, representing a 2.7% R3X read-through rate.

TABLE 2
Comparison of the Read-through Activity
for the Usher Mutations USH2A and USH1F

| | | USH2A Read- | USH1F Read- |
|---|---|---|---|
| Compound | NB Name | through (Max RT) | through (Max RT) |
| ELX-10 | NB157 | 22.6 | 18.7 |
| ELX-06 | NB128 | 15.5 | 10.5 |
| ELX-01 | NB122 | 14.6 | 11.9 |
| ELX-07 | NB124-MeS | 14.4 | 11.0 |
| ELX-03 | NB118 | 12.7 | 11.4 |
| ELX-04 | NB127 | 11.9 | 9.8 |
| Gentamicin | | 5.81 | 5.02 |

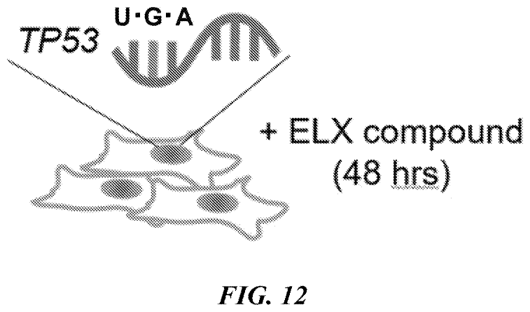

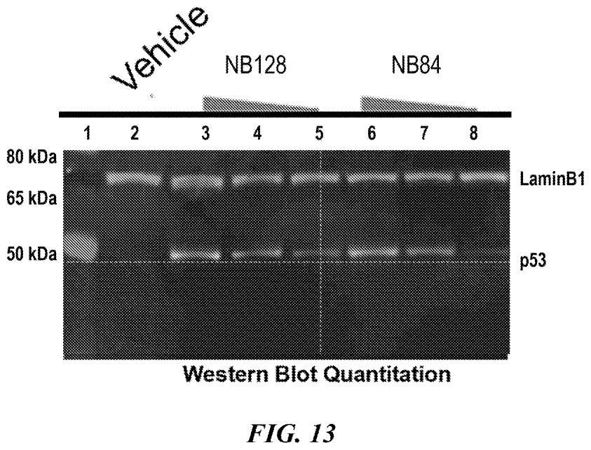

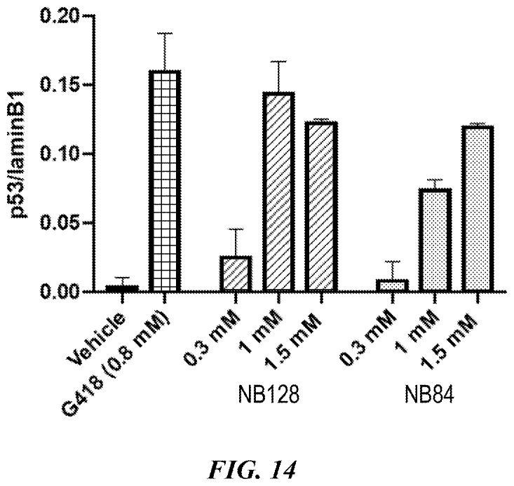

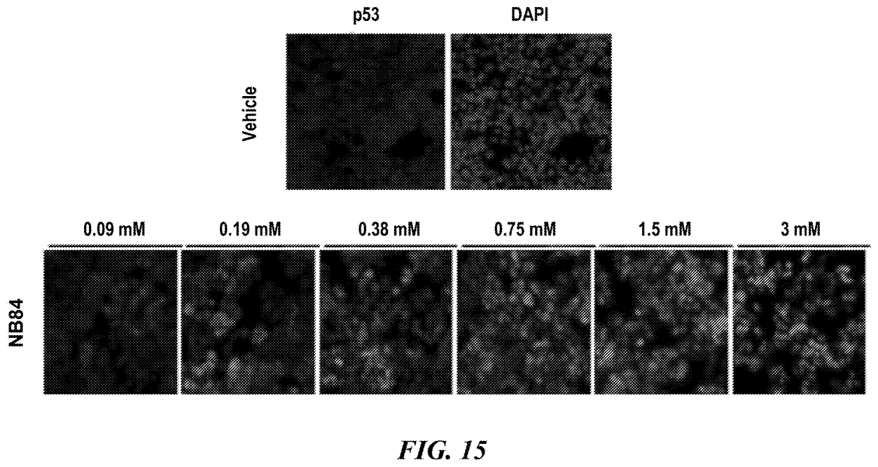

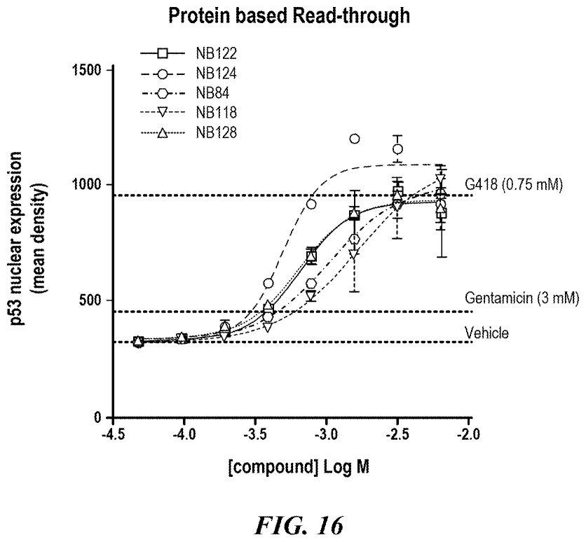

Protein Based Read-through Assay. NB122, NB124, NB84, NB118, and NB128 were evaluated for their protein-based read-through potential in DMS114 cells, which harbor a native R213X nonsense mutation in human TP53 as depicted in the scheme shown in FIG. 12. FIG. 13 shows representative Western blots for the detection of full length p53 after administration of NB128 and NB84. Detection was quantified in comparison to LaminB1 loading control by densitometry as shown in FIG. 14. A high-throughput immunofluorescence assay measuring p53 localized at the nucleus is shown in FIG. 15 with dose-dependence studies for NB84. Lastly, FIG. 16 provides full dose response curves for NB122, NB124, NB84, NB118, and NB128 with the error bars representing a standard deviation (SD). Taken together, these data confirm that NB122, NB124, NB84, NB118, and NB128 induce dose-dependent nonsense mutation read-through as measured by protein expression.

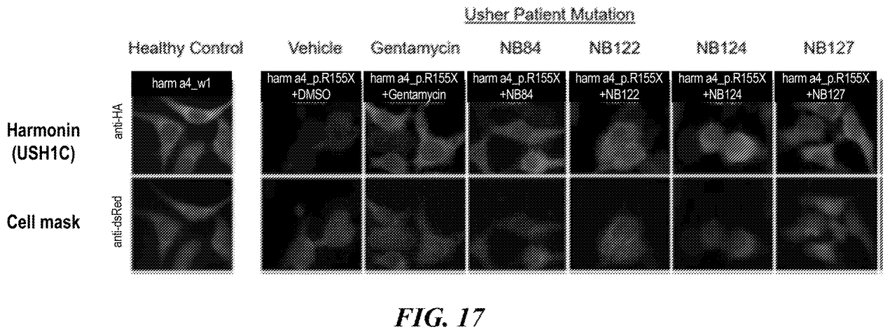

The ability of these compounds to induce protein expression of nonsense mutations associated with Usher syndrome was evaluated using USH1C nonsense mutations. NB84, NB122, NB124, and NB127 were evaluated for read-through of the USH1C nonsense mutation R155X, and as shown in FIG. 17 all four compounds increased protein expression versus gentamicin and vehicle control.

### Example 3: Read-Through Activity Following Intravitreal Administration

Read-through activity following intravitreal injection of aminoglycosides was evaluated in animal models. Intravitreal injection to the posterior segment (i.e., back of eye) in mice models was found to induce significant read-through activity of nonsense mutations associated with Usher Syndrome. Thus, the tested aminoglycosides not only exhibit a superior tolerability profile as compared to conventional aminoglycosides such as gentamicin, but also exhibit significantly improved read-through activity.

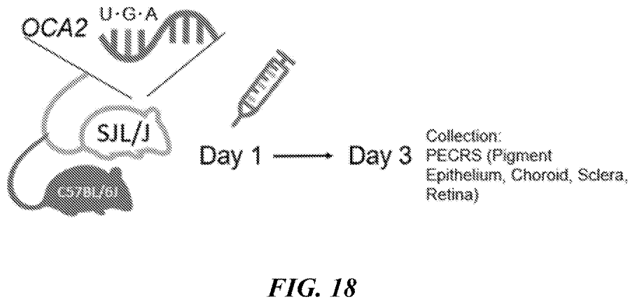

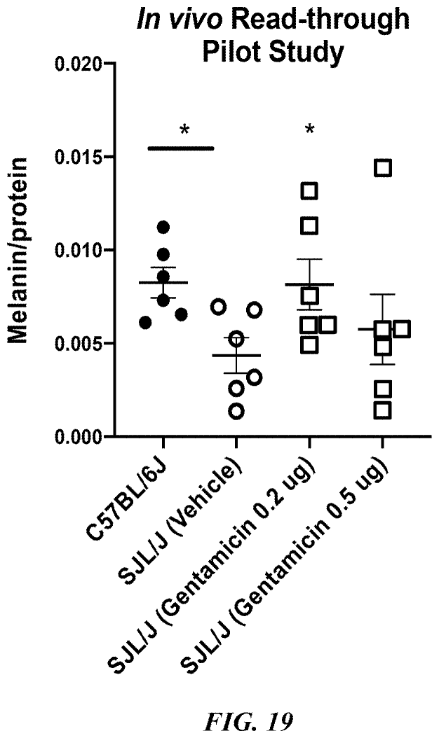

Albino mice (SJL/J mice) harbor the nonsense mutation R262X in the Oca2 gene resulting in an absence of melanin production in melanocytes. This model provides an initial evaluation of compound availability to the back of the eye when administered by intravitreal injection—higher concentrations of melanin correspond to greater read-through activity. FIG. 18 shows a schematic of the intravitreal injection of the SJL/J mice. Pilot studies with gentamicin to identify a reference control condition as shown in FIG. 19 demonstrated that 0.2 μg gentamicin significantly (i.e., t-test vs. vehicle control) increases melanin, with toxicity observed at 0.5 μg.

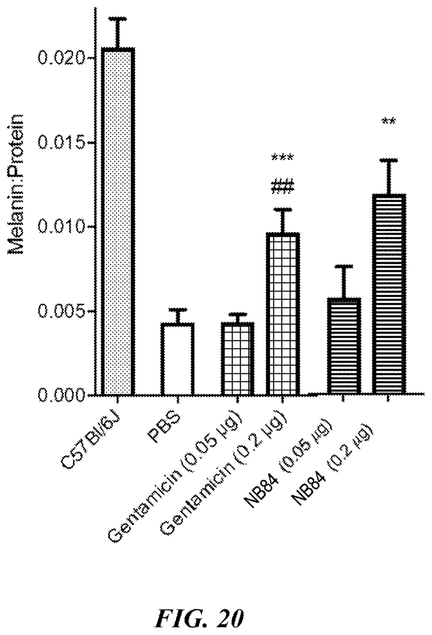

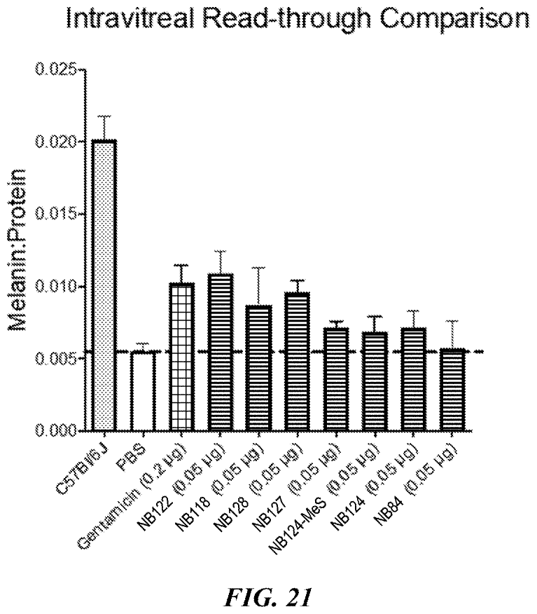

NB122, NB124, NB124-MeS, NB84, NB118, NB127, NB128, and NB157 were evaluated for read-through activity following intravitreal injection into SJL/J mice. FIGS. 20-21 show intravitreal read-through for each compound relative to gentamicin. These data demonstrate that there is a dose-dependent increase in melanin production in the eye upon administration of NB122, NB124, NB124-MeS, NB84, NB118, NB127, NB128, and NB157, indicating that these compounds are capable of inducing read-through activity in the eye following intravitreal administration.

From the foregoing, it will be appreciated that specific embodiments of the invention have been described herein for purposes of illustration, but that various modifications may be made without deviating from the scope of the invention. Accordingly, the invention is not limited except as by the appended claims.

Para. A. A method of treating an ocular disease associated with one or more nonsense mutations in a subject, comprising administering to said subject a therapeutically effective amount of one or more aminoglycosides selected from the group consisting of NB30, NB54, NB84, NB118, NB122, NB124, NB124-MeS, NB127, NB128, and NB157.

Para. B. A method of treating ocular disease associated with one or more nonsense mutations in a subject, comprising administering to said subject a therapeutically effective amount of one or more aminoglycosides, wherein said one or more nonsense mutations are selected from the group consisting of R3X (PCDH11), R155X (USH1C), R245X (PCDH15), and R626X (USH2A).

Para. C. A method of treating an ocular disease associated with one or more nonsense mutations in a subject, comprising intravitreally administering to said subject a therapeutically effective amount of one or more aminoglycosides.

Para. D. A method of treating an ocular disease associated with one or more nonsense mutations in a subject, comprising intravitreally administering to said subject a therapeutically effective amount of one or more aminoglycosides, wherein the one or more aminoglycosides induces read-through activity without exceeding the safety exposure threshold of the one or more aminoglycosides.

Para. E. A method of treating Usher Syndrome in a subject in need thereof, comprising administering to said subject a therapeutically effective amount of one or more aminoglycosides.

Para. F. The method of any one of Para. A and C-E, wherein the subject has a R3X, R245X in the PCDH11 gene or a R155X nonsense mutation of the USH1C gene.

Para. G. The method of any one of Para. A and C-F, wherein the subject has a R626X nonsense mutation of the USH2A gene.

Para. H. The method of any one of Para. A-G, wherein the subject is administered about 0.3 mg/kg to about 2.5 mg/kg of the one or more aminoglycosides.

Para. I. The method of any one of Para. A, B, or E, wherein the one or more aminoglycosides are administered by intravitreal injection.

Para. J. The method of any one of Para. A-I, wherein the one or more aminoglycosides is formulated into a pharmaceutical composition.

Para. K. The method of Para. J, wherein the pharmaceutical composition further comprises one or more pharmaceutically acceptable carriers.

Para. L. The method of any one of Para. A-D, wherein the ocular disease associated with one or more nonsense mutations includes an inherited retinal disease, retinitis pigmentosa, Usher Syndrome, Stickler Syndrome, aniridia, Leber congenital amaurosis, and choroideremia.

Para. M. A method of increasing gene expression or gene read-through in a retinal cell comprising contacting the retinal cell with one or more aminoglycosides, or a pharmaceutical formulation comprising one or more aminoglycosides, wherein the gene expression or the gene read-through is adversely affected by one or more nonsense mutations.

Para. N. The method of Para. M, wherein the cell is contacted ex vivo.

Para. O. The method of Para. M, wherein the cell is contacted in vivo.

Para. P. The method of any one of Para. M-O, wherein the one or more aminoglycosides is selected from the group consisting of a Category I compounds, Category II compounds, Category III compounds, Category IV compounds, and Category VI compounds.

Para. Q. The method of any one of Para. M-P, wherein the one or more aminoglycosides include NB30, NB54, NB84, NB118, NB122, NB124, NB124-MeS, NB127, NB128, and NB157.

Para. R. A formulation comprising one or more aminoglycosides for treating an ocular disease associated with one or more nonsense mutations in a subject.

Para. S. A formulation comprising one or more aminoglycosides for treating an ocular disease associated with one or more nonsense mutations in a subject, wherein the formulation is for intravitreal administration.

Para. T. The formulation of Para. R or S, wherein the formulation is a pharmaceutical formulation.

Para. U. The formulation of Para. T, further comprising one or more pharmaceutically acceptable carriers.

Para. V. The formulation of any one of Para. R-U, wherein the one or more aminoglycosides is selected from the group consisting of a Category I compounds, Category II compounds, Category III compounds, Category IV compounds, and Category VI compounds.

Para. W. The formulation of any one of Para. R-V, wherein the one or more aminoglycosides thereof include NB30, NB54, NB84, NB118, NB122, NB124, NB124-MeS, NB127, NB128, and NB157.

Para. X. A kit comprising one or more aminoglycosides for use in treating an ocular disease associated with one or more nonsense mutations in a subject or for use in increasing gene expression in a cell.

Para. Y. The kit of Para. X, further comprising instructions for use.

Para. Z. The kit of Para. X or Y, wherein the one or more aminoglycosides is selected from the group consisting of a Category I compounds, Category II compounds, Category III compounds, Category IV compounds, and Category VI compounds.

Para. AB. The kit of Para. X-Z, wherein the one or more aminoglycosides include NB30, NB54, NB84, NB118, NB122, NB124, NB124-MeS, NB127, NB128, and NB157.

Para. AC. One or more aminoglycosides for use in formulating a medicament for use in treating an ocular disease associated with one or more nonsense mutations in a subject.

Para. AD. The one or more aminoglycosides of Para. AC, wherein the one or more aminoglycosides and/or derivatives thereof is selected from the group consisting of a Category I compounds, Category II compounds, Category III compounds, Category IV compounds, and Category VI compounds.

Para. AE. The one or more aminoglycosides of Para. AC or AD comprising NB30, NB54, NB84, NB118, NB122, NB124, NB124-MeS, NB127, NB128, and NB157.

Para. AF. A method of treating an ocular disease associated with one or more nonsense mutations in a subject, comprising intravitreally administering to said subject a therapeutically effective amount of one or more aminoglycosides selected from the group consisting of NB30, NB54, NB84, NB118, NB122, NB124, NB124-MeS, NB127, NB128, and NB157, wherein said one or more nonsense mutations are selected from the group consisting of R3X (PCDH11), R155X (USH1C), R245X (PCDH15), and R626X (USH2A).

### REFERENCES

1. Goldmann et al. EMBO Mol Med 4(11):1186-1199 (Nov. 2012)
2. Möller et al. 2016 The Annual Meeting of the Association for Research in Vision and Ophthalmology (ARVO)
3. Nudelman et al. Bioorg Med Chem 18(11):3735-3746 (Jun. 1, 2010)
4. Kandasamy et al. J Med Chem 55(23):10630-10643 (Dec. 13, 2012)
5. Mashima et al. J Natl Cancer Inst 97(1); 765-777 (May 18, 2005).
6. Shoji et al., Exp Anim 64(2): 171-179 (Jan. 22, 2015).

1. (canceled)

2. A method of treating an ocular disease associated with one or more nonsense mutations in a subject, comprising intravitreally administering to said subject a therapeutically effective amount of one or more aminoglycosides selected from the group consisting of NB118, NB122, NB124, NB124-MeS, NB127, NB128, and NB157.

3. The method of claim 2, wherein the subject has one or more nonsense mutations selected from the group consisting of R3X in the PCDH11 gene, R245X in the PCDH11 gene, R155X in the USH1C gene, and R626X in the USH2A gene.

4. (canceled)

5. The method of claim 2, wherein the subject is administered about 0.3 mg/kg to about 2.5 mg/kg of the one or more aminoglycosides.

6. The method of claim 2, wherein the one or more aminoglycosides are administered by intravitreal injection.

7. The method of claim 2, wherein the one or more aminoglycos ides are formulated into a pharmaceutical composition.

8. The method of claim 7, wherein the pharmaceutical composition further comprises one or more pharmaceutically acceptable carriers.

9. The method of claim 2, wherein the ocular disease associated with one or more nonsense mutations includes an inherited retinal disease, retinitis pigmentosa, Usher Syndrome, Stickler Syndrome, aniridia, Leber congenital amaurosis, and choroideremia.

10. The method of claim 2, wherein said one or more nonsense mutations are selected from the group consisting of R3X (PCDH11), R155X (USH1C), R245X (PCDH15), and R626X (USH2A).

11-13. (canceled)

14. A formulation for use in treating an ocular disease associated with one or more nonsense mutations in a subject, comprising one or more aminoglycosides selected from the group consisting of NB118, NB122, NB124, NB124-MeS, NB127, NB128, and NB157, wherein the formulation is for intravitreal administration.

15. (canceled)

16. The formulation of claim 14, wherein the formulation further comprises one or more pharmaceutically acceptable carriers.

17. (canceled)

18. A kit for use in treating an ocular disease associated with one or more nonsense mutations in a subject, comprising one or more aminoglycos ides selected from the group consisting of NB118, NB122, NB124, NB124-MeS, NB127, NB128, and NB157.

19. The kit of claim 18, further comprising instructions for use.

20-22. (canceled)

23. The method of claim 9, wherein the ocular disease associated with one or more nonsense mutations is Usher Syndrome.
